(19) United States
(12) Patent Application Publication
(10) Pub. No.: US 2025/0253031 A1
(43) Pub. Date: Aug. 7, 2025

(54) ACTIVELY INFLUENCING A STATE OF A USER OF A COMPUTING DEVICE

(71) Applicant: Nvidia Corporation, Santa Clara, CA (US)

(72) Inventor: Thorsten Stremlau, Cary, NC (US)

(21) Appl. No.: 18/435,131

(22) Filed: Feb. 7, 2024

(51) Int. Cl. G16H 20/70; A61B 5/0205; A61B 5/0533; A61B 5/16

(52) U.S. Cl. CPC G16H 20/70; A61B 5/0205; A61B 5/0533; A61B 5/163; A61B 5/165

(57) ABSTRACT

Approaches presented herein provide for automatic detection and remediation of various undesired states or behaviors of a user using an electronic device. Embodiments allow for detection of high levels of anxiety (as might be associated with post-traumatic stress disorder (PTSD)) of the user and initiating calming mechanisms to attempt to reduce a level of anxiety in the user. Aspects of the user can be monitored and analyzed to determine a level of anxiety, stress, etc., of the user. Such analysis can be performed using a machine learning model that is trained to infer a level of anxiety of a user based on a variety of possible inputs. If the user is determined to likely be in a high anxiety state, one or more calming mechanisms or adjustments can be made automatically in order to attempt to reduce the level of anxiety being experienced by the user.

### BACKGROUND

There is an increasing focus and understanding of the mental and emotional health of individuals. There is a corresponding increase in the diagnosis of individuals with mental health issues, such as post-traumatic stress disorder (PTSD) or other such anxiety disorders. In order to attempt to minimize the impact of a given environment, such as the workplace, on the mental state or behavior of a user, prior approaches typically required a person to indicate that they have, or may be subject to, such issues, states, or behaviors, and then the user could be placed in a location that might be better suited for the user. This might include placing the person in a quiet corner location, keeping the person away from windows where a lot of activity occurs or where others can see the person, etc. Such factors are not dynamic in nature and cannot be automatically adjusted based on a current state of the user. Further, some of these factors may require others to have knowledge of the potential issues, or be able to discern the issues, which may exacerbate the situation and decrease the comfort level of the person already experiencing stress, anxiety, or other forms of discomfort.

### BRIEF DESCRIPTION OF THE DRAWINGS

Various embodiments in accordance with the present disclosure will be described with reference to the drawings, in which:

FIG. 1 illustrates an example view of a person using an electronic device with one or more sensors that are able to capture data reflective of a current state of the user, according to at least one embodiment;

FIGS. 2A and 2B illustrate aspects of a user interface (UI) that can be modified based in part on determined user state or behavior, according to at least one embodiment;

FIG. 3 illustrates components of an example computing device that can capture information about a user and then modify operation based in part upon analyzing that information, according to at least one embodiment;

FIG. 4 illustrates components of an example computing framework, according to at least one embodiment;

FIGS. 5A, 5B, and 5C illustrate example processes for determining a current state or behavior of a person and taking one or more actions based in part thereon, according to at least one embodiment;

FIG. 6 illustrates components of a distributed system that can be utilized to monitor a state or behavior of a person and modify one or more operational parameters based in part upon the state or behavior, according to at least one embodiment;

FIG. 7A illustrates inference and/or training logic, according to at least one embodiment;

FIG. 7B illustrates inference and/or training logic, according to at least one embodiment;

FIG. 8 illustrates an example data center system, according to at least one embodiment;

FIG. 9 illustrates a computer system, according to at least one embodiment;

FIG. 10 illustrates a computer system, according to at least one embodiment;

FIG. 11 illustrates at least portions of a graphics processor, according to one or more embodiments;

FIG. 12 illustrates at least portions of a graphics processor, according to one or more embodiments;

FIG. 13 is an example data flow diagram for an advanced computing pipeline, in accordance with at least one embodiment;

FIG. 14 is a system diagram for an example system for training, adapting, instantiating and deploying machine learning models in an advanced computing pipeline, in accordance with at least one embodiment; and

FIGS. 15A and 15B illustrate a data flow diagram for a process to train a machine learning model, as well as client-server architecture to enhance annotation tools with pre-trained annotation models, in accordance with at least one embodiment.

### DETAILED DESCRIPTION

In the following description, various embodiments will be described. For purposes of explanation, specific configurations and details are set forth in order to provide a thorough understanding of the embodiments. However, it will also be apparent to one skilled in the art that the embodiments may be practiced without the specific details. Furthermore, well-known features may be omitted or simplified in order not to obscure the embodiment being described.

The systems and methods described herein may be used by, without limitation, non-autonomous vehicles, semi-autonomous vehicles (e.g., in one or more advanced driver assistance systems (ADAS)), piloted and un-piloted robots or robotic platforms, warehouse vehicles, off-road vehicles, vehicles coupled to one or more trailers, flying vessels, boats, shuttles, emergency response vehicles, motorcycles, electric or motorized bicycles, aircraft, construction vehicles, trains, underwater craft, remotely operated vehicles such as drones, and/or other vehicle types. Further, the systems and methods described herein may be used for a variety of purposes, by way of example and without limitation, for machine control, machine locomotion, machine driving, synthetic data generation, model training or updating, perception, augmented reality, virtual reality, mixed reality, robotics, security and surveillance, simulation and digital twinning, autonomous or semi-autonomous machine applications, deep learning, environment simulation, object or actor simulation and/or digital twinning, data center processing, conversational AI, generative AI with large language models (LLMs), light transport simulation (e.g., ray-tracing, path tracing, etc.), collaborative content creation for 3D assets, cloud computing and/or any other suitable applications.

Disclosed embodiments may be comprised in a variety of different systems such as automotive systems (e.g., a control system for an autonomous or semi-autonomous machine, a perception system for an autonomous or semi-autonomous machine), systems implemented using a robot, aerial systems, medial systems, boating systems, smart area monitoring systems, systems for performing deep learning operations, systems for performing simulation operations, systems for performing digital twin operations, systems implemented using an edge device, systems incorporating one or more virtual machines (VMs), systems for performing synthetic data generation operations, systems implemented at least partially in a data center, systems for performing conversational AI operations, systems for performing generative AI operations using LLMs, systems for performing light transport simulation, systems for performing collaborative content creation for 3D assets, systems implemented at least partially using cloud computing resources, and/or other types of systems.

Approaches in accordance with various illustrative embodiments provide for the active monitoring and influencing of the behavior and/or state of a user. In particular, various embodiments allow for the detection and/or monitoring of anxiety or stress (such as may be associated with post-traumatic stress disorder (PTSD)) in users, as well as the ability to initiate mitigating (e.g., calming) mechanisms to attempt to reduce a level of anxiety or stress, or otherwise influence or improve an observed state, in those users. Taking such an approach, aspects of a user can be monitored by a computing device, for example, and those aspects can be used to determine a level of stress, anxiety, or other such state or behavior of a user. This may include, for example, analyzing the pulse (including both heart rate and variability) and blood pressure determinable from images (or video) captured using a standard integrated camera, or using other sensor data to detect pupil dilation, changes in voice tone or pitch, respiratory rate or variability, variations in user input, or galvanic skin response, for example. This information can then be used to determine a level of anxiety, current state, or observed behavior of the user based on analyzing that information. In at least one embodiment, such analysis can be performed using a machine learning model that is trained to infer aspects such as a level of anxiety of a user, for example, based on a variety of possible inputs. If the user is determined to likely be in a high anxiety state, for example, one or more calming mechanisms or other such mitigating adjustments can be made automatically in order to attempt to reduce the level of stress or anxiety being experienced by the user. These adjustments can include, for example, reducing a brightness or glare of a display, adjusting a color scheme of a display to more calming colors, changing a background image or digital wallpaper to present a calming or serene scene, reducing a number, frequency, or type of notifications or messages received by the user until the level of stress is reduced, reducing a strength or abrupt occurrence of haptic feedback, providing haptic feedback to discretely communicate current user state, recommending calming exercises, automatically activating a “do not disturb” function, or playing calming sounds or music, among other such adjustments. In at least one embodiment, such functionality can be provided in a chip or platform that can be included in a device and then leveraged by applications running on that device. In at least one embodiment, “passive” data may be preferred over active data since the user is not required to perform any actions and can simply be monitored, such as by using a front-facing camera of a laptop. Although the focus of the disclosure is primarily toward mitigation, certain action or access controls could be implemented as well based in part upon detected user state.

Variations of this and other such functionality can be used as well within the scope of the various embodiments as would be apparent to one of ordinary skill in the art in light of the teachings and suggestions contained herein.

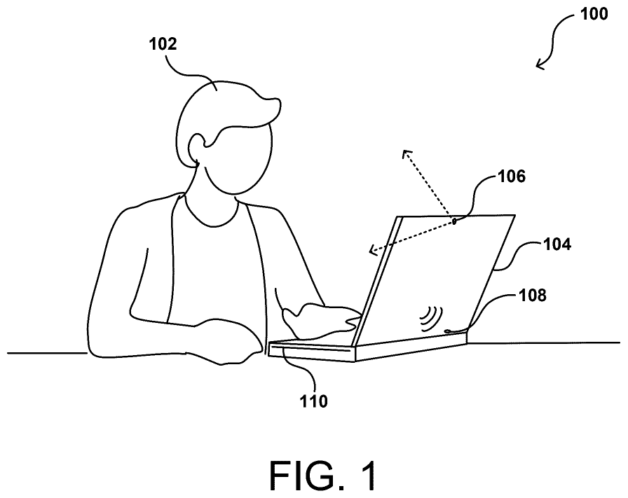

FIG. 1 illustrates an example arrangement 100 of a user 102 interacting with an electronic device, in this case a human user interacting with a notebook computer 104. A human user may interact with many other such electronic devices to perform a variety of tasks. For many of these devices, there will be one or more sensors that are able to capture input with respect to a user, whether that input is explicitly or implicitly provided by the user 102, or whether that input may result from monitoring or capturing data that may be indicative of a current state or behavior of the user 102. As an example, the notebook computer can have explicit input mechanisms, such as a keyboard, mouse, touchpad, or touchscreen, allowing the user to provide specific input. The notebook computer 104 can also have at least one front-facing camera 106 that can capture image data of a user when that user is within a field of view of that camera, and a microphone 108 for capturing audio that may include audio generated by the user 102. This audio may include speech uttered by the user 102, grunts or groans emitted by the user, breathing sounds generated by the user, or sounds made by the user interacting with a device or object, such as rolling a chair back and forth or tapping a pencil. This example notebook computer 104 also includes one or more touch or pressure sensitive elements 110 which are able to detect a rate, length, and/or amount of pressure applied by the user to various elements of the notebook computer 104, such as keys of a keyboard or a touch screen. Various other sensors may be used to capture data representing a state or behavior of the user as well within the scope of the various embodiments.

Data regarding a state or behavior of the user can be used to determine when or how to perform a number of tasks, such as to reduce the brightness of a display if a user is not looking in the direction of the notebook computer 104, or to put the notebook computer 104 in a low power mode if the user is determined to be away from the device for a period of time. Approaches in accordance with various embodiments attempt to utilize at least some of this sensor data, and other available data, to attempt to determine a mental or physical state of the user. These approaches can then determine whether to make any adjustments, or take any actions, based at least in part upon a determined state or determined behavior of a user. For certain states or behaviors, this may include taking actions to attempt to mitigate symptoms, modify the state, or adjust the behavior of the user in some way.

In at least one embodiment, an attempt can be made to identify a user who is exhibiting signs of anxiety. There is an increase in users with anxiety disorders such as, but not limited to, post-traumatic stress disorder (PTSD). When a user is experiencing a high level of anxiety, it can be beneficial to attempt to reduce the level of anxiety experienced. Approaches in accordance with various embodiments can attempt to modify the operation of an electronic device, such as a notebook computer 104, in an attempt to calm the user 102 or otherwise reduce anxiety, or symptoms of anxiety. The anxiety level of the user can be monitored during this time, to determine whether the changes are having a positive impact on the anxiety level of the user. Adjustments or further actions can be taken as necessary based on changes (or lack of changes) in state, and once the user returns to a relatively normal state (or at least has at most an acceptable level of anxiety) for a period of time, then the changes or modifications may be slowly undone, or reverted back to prior operational state of the notebook computer 104, while in some embodiments the changes may remain in place throughout the duration of a current session. In at least some embodiments, the user may have control over the number or types of actions that are taken, as well as the level of anxiety at which specific actions may be taken, among other such options. Further, although discussed with respect to anxiety, there may be various other emotional, mental, or physical states or behavior that can be detected and managed as well, as may include depression, anger, sadness, fatigue, and the like. The actions taken can also vary by the type or extent of behavior or state that is identified for a given user, and may also vary for different users or types of users.

In at least one embodiment, a current anxiety level of a user (or other person or subject (e.g., a pet dog) within a capture or detection range of a device) can be determined based at least in part on image data captured of at least an eye or facial region of the user. This can include analyzing captured image data, and potentially cropping or otherwise processing the image data for analysis, such as to adjust a color depth, reduce a presence of noise, or modify the brightness or contrast for maximum detectability. The processed image data can then be analyzed to attempt to determine physical aspects of a user. In at least one embodiment, a sequence of captured images or video frames can be analyzed to attempt to determine the pulse and/or blood pressure of the user. This can include performing transdermal optical analysis of the image data. Variations in the image data using such analysis can be used to determine information such as systolic pressure, diastolic pressure, and pulse pressure values using blood flow detected in the face of the user by analyzing the captured image data. The image data can be analyzed to provide values for respiratory rate, heart rate and blood oxygen/saturation levels as well. In at least one embodiment, a machine learning model can be trained to infer any or all of these and other such values from a stream or sequence of input images, image data, or video frames.

In at least one embodiment, the determined state or behavior of the user may be of a type or extent for which at least one remedial action can be taken automatically. For different types of states or behaviors, there may be different acceptable ranges or types, as well as thresholds or criteria at which one or more remedial actions should be taken. As used herein, a remedial action can be any action or operation that may be taken to attempt to modify a state, behavior, or aspect of a user, such as to calm a user determined to be having an anxiety attack. The acceptable ranges or values, or thresholds or states at which remedial action should be taken, can be user configurable and/or learned over time, and may be customized for individual users. For at least some users, a baseline or set of expected values may be determined for various circumstances, such that unacceptable variances can be determined with respect to these baselines, which may vary naturally between different users. In at least one embodiment, or for at least certain states or behaviors, a user may be required to opt in to monitoring and/or remediation, in order to comply with any requirements or regulations, in addition to any personal preferences.

Depending in part upon the type of state or behavior, as well as personal preferences or other such factors, any adjustments or actions can be taken discretely, in order to prevent the adjustments or actions from being obvious to others (e.g., person or subject) in the vicinity, for example, as well as to not be jarring to the person at issue, which may otherwise worsen (e.g., produce an undesirable effect on) their state, behavior, symptoms, and/or anxiety level in certain situations. For example, if brightness values are to be changed or color schemes modified, there can be a transition period where the values are slowly and gently modified over a period of time, instead of a quick switch between values, schemes, or states. For another example, if music is to be played, the music can start at a very low volume and slowly increase the volume, etc.

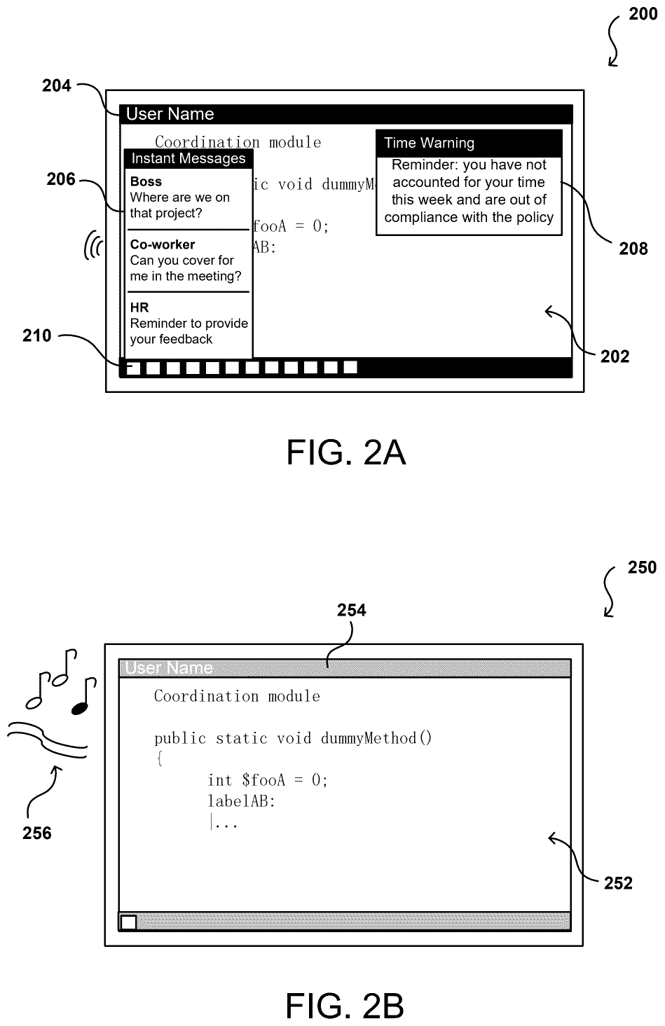

FIGS. 2A and 2B illustrate aspects of a user interface (UI) that can be modified automatically in response to detecting or identifying a specific state or behavior of a user. In at least one embodiment, the modifications (or extent of the modifications) made may be determined at least in part upon the type of user state or behavior, as well as the extent of that state or behavior. As mentioned, the types or extents of actions or modifications to be made may also be configurable, such as by the person at issue, an employer, a therapist, and the like.

FIG. 2A illustrates a first view 200 of a user interface on a computing device that may be displayed under “normal” or default conditions, such as for a typical user or a user who is not determined to be in an undesired state. This example interface may have various interface elements 204, such as title bars, task bars, and the like, which may be displayed according to a default color scheme. The interface may include icons 210 in the task bar that indicate which programs or applications are open or active on the computing device. The interface also includes additional windows, panels, or pop-up elements, such as an instant messaging overlay interface 206 that may pop up or come to the forefront whenever a message is received, as well as a popup warning window or panel 208 that may convey any important or urgent information. Various other elements can be included or made active in such an interface as well within the scope of various embodiments.

For a user who suffers from anxiety or depression, for example, at least some of these aspects may have negative impacts on the mental state of a user. Other aspects may not have negative impact necessarily but can be adjusted in a way that can help improve the mental state of the user. For example, popup warnings 208 or notifications may be used to indicate certain types of information, such as where the user has failed to do something or is behind in a task, or that there is a something that has come up that needs the user's attention. In this example, the warning indicates that the user has fallen behind in time entry and needs to address the task. If a user is experiencing anxiety or stress, which may be a factor of the pressure on the user or number of tasks the user feels responsible for, this additional pressure (particularly in this format) may cause increased stress or anxiety for the user. At least during a period of high anxiety or stress, the system may prevent or delay the display of such messages, or may at least present the information in less triggering ways, such as to send an email message or wait until the user is back out of an undesired state. There may be certain emergency warnings, such as a fire in the building, that may be displayed no matter what due to safety or other such concerns; but, for non-critical messages or alerts, the presentation can be modified during a period of undesired state of a user.

Similarly, the instant messaging window 206 might pop up over the interface or primary window 202 when a message is received, and in some instances a sound will be generated or played indicating the receipt of such a message. The receipt of such messages, particularly high numbers of these messages over a short period of time, can also be triggering for certain users in certain undesired states. In at least one embodiment, the presentation of such messages can be modified. For example, a number of messages displayed can be reduced, and/or the sounds typically played for the receipt of a message can be temporarily suspended, changed, or played at lower volume. In at least one embodiment, the receipt of a message may not cause the message window to pop up while a user is in an undesired state and may stay hidden behind the primary window 202 unless accessed by the user. In some embodiments, the types of messages can be managed for a period of time, such as to only display messages from certain senders or hide messages including certain triggering words (such as late, deadline, missed, angry, etc.). In some embodiments, these messages can be queued or cached and can be presented at no more than an allowable rate, such as at no more than one message every five minutes.

Similarly, a user feeling overwhelmed may be triggered by the number of concurrent open applications on the device. In order to provide a sense of reduced obligation or fewer concurrent demands, the interface may hide the taskbar or may reduce the number of icons 210 displayed, such as to only illustrate an icon for the active application or interface 202 in which the user is working or engaging. Various other notifications (such as for email messages, voice mail messages, or new tasks) can be modified as well, such as to limit a visibility or frequency, hide at least certain types of content for a period of time, and so forth.

Other actions may be taken that may not be directly related to the types of content to be presented through such an interface, but that may have a calming or soothing effect on at least some users. As an example, the interface view 250 of FIG. 2B illustrates, in addition to the changes discussed above, changes to the appearance or presentation of the interface presented to the user. In this example, a color scheme used for the interface has been modified, wherein colors believed to have “calming” effects can be used for certain elements 254 of the interface. This may include reducing a contrast or brightness of colors, for example, but may also include using colors in a blue or pastel palette rather than one with reds and yellows. As illustrated, other potentially distracting or triggering elements have been removed or reduced, so the user can focus on the primary task through a primary (or in-focus) window 252 or interface. Reducing the clutter, number of distractions, and potential interruptions, along with presenting more calming colors, can help to reduce the amount of potentially stress-inducing content being conveyed to the user over a period of time, which can hopefully help to reduce the level of stress, anxiety, or other undesired state, and hopefully have a calming effect on the user.

Other specific actions can be taken to attempt to further calm the user or to help reduce or mitigate the undesired state of the user. For example, calming music 256 can be played, at a reasonably low volume, which can attempt to soothe the user or place the user in a calmer mental state. The user can specify types of music, or music believed to have calming effects can be played, among other such options. In some embodiments, notifications may be displayed to the user to ask the user to take specific actions, such as to meditate for a period of time, take deep breaths, etc. In some embodiments, such instructions or request may be provided through audio speech (recorded or synthesized) played through speakers or headphones. In certain situations where it may not be possible to play music, or where the user does not want to bring attention to the current undesired state, other approaches can be used to convey information, such as through haptic feedback. For example, a device separate but communicatively coupled with the notebook computer 104 (e.g., a mouse or chair), with haptic feedback capability, can be triggered to provide haptic feedback, such as by using a specific haptic pattern, in response to the user being determined to be in an undesired state. For example, if it is determined that the user is likely having an anxiety attack or at risk of an anxiety attack, then haptic feedback can be provided to the user to indicate or signal that certain action should be taken to prevent or mitigate such a state. This may be a signal for a user to step away, if possible, or to take another action based in part upon the type or level of undesired state.

In at least one embodiment, the sensor data can be analyzed over time to determine whether the state or behavior is improving, such as where a state level is decreasing. If the state level is improving, then the remedial actions can continue, at least until such time as the user is back within an acceptable state or level, and in some instances back for at least for a minimum period of time. If the state is not improving, or is getting worse, then at least one different action can be taken, or an extent of an action can be changed. For example, if the anxiety level of a user is still increasing, then the number or frequency of messages shown can be further reduced or removed for a period of time. If notifications are able to be displayed, then notifications can be selected that may be more direct or that provide different recommendations, such as to step away, breathe, etc. In extreme situations, and where allowed and/or permitted, a notification or alarm might be raised, which may be directed to the user and potentially another entity, such as a doctor, hospital, caregiver, spouse, nurse, or other designated person. In environments or locations where there may be other smart devices that can receive instructions, then other actions may be taken such as to dim the lights in the room, lower the shades or close the blinds, play soft music, prevent doorbell notifications, activate a massaging function in a chair, etc.

In at least one embodiment, the actions taken and reactions determined for a given user, type of user, or set of users can be monitored and analyzed over time. This information can be used to determine which actions or modifications work or do not work to mitigate certain states or behaviors in these users, as well as the extents to which various actions, or combinations of actions, are determined to work. This can also be analyzed for specific users, as well as specific undesired states or behaviors. For example, it can be learned that a first set of actions might work well for a first user exhibiting a first type of undesired behavior, while a different set of actions might work for a second user or a second type of undesired behavior. Further, different actions might have different effects at different locations. For example, a person at home might react better to certain actions than if that person is at work or at a friend's house, for example, as the user may not want others to discover that the user is in a specific state, or there may be other factors at that location that are impacting the state. For example, the noise of a neighbor might be causing stress at home such that calming music to drown out the noise might help, while the user would not want that calming music played at a given volume at work as that might notify co-workers in the area that the user is in an undesired state, or might at least be distracting or annoying to co-workers nearby, which might in turn be a source of additional stress to a user. If machine learning is used to infer the best actions, or combinations of actions, to take under a given set of circumstances, then the machine learning model can undergo further training using the actions and responses to provide for better inferences under a variety of different circumstances.

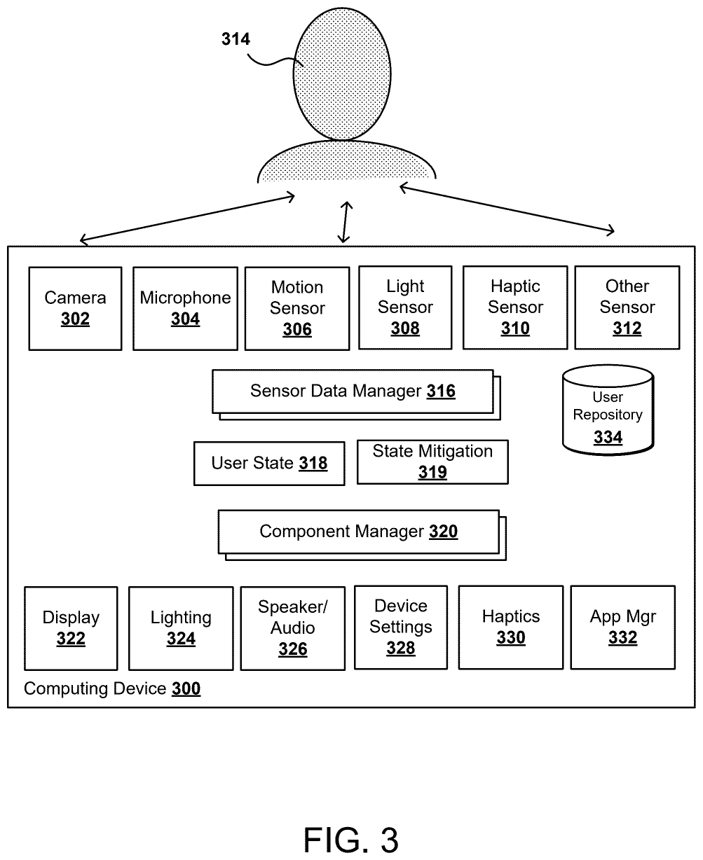

FIG. 3 illustrates components of an example computing device 300 that can monitor a state or behavior of a user 314, subject, or person within a detectable proximity, and take one or more actions to attempt to improve and/or modify that state or behavior. While an example set of components is illustrated that are all part of the computing device 300, it should be understood that there may be additional, fewer, or alternative components in other examples, and that some of these components may be located remote from, but in direct or indirect communication with, the computing device 300.

In this example, the computing device 300 includes a number of sensors or components that can capture or obtain data about a current state or behavior of a user. This can include, for example, at least one camera 302 that is able to capture image and/or video information about the user. This may be an ambient light camera to capture aspects such as facial expressions, movements, or color variations in the skin of a user, for example, or may be an infrared camera to capture information about the pupils of the user, among other such options. A computing device 300 might include a microphone 304 to capture sounds associated with the user, such as uttered speech or sounds, breathing sounds, etc., and at least one motion or depth sensor 306 to detect an amount, type, or pattern of motion of the user (or objects associated with the user). The computing device might include a light sensor 308 that is able to determine an amount of ambient light in the area, such as to determine whether the user is sitting in the dark or in bright light, and may include a haptic sensor 310 (or other pressure or touch sensor) to determine variations in pressure being applied by the user, variations or irregularities in frequency or rate of input, or other variations in patterns of user input or behavior. There may be various other sensors 312 that can capture potentially relevant information as well within the scope of the various embodiments.

In this example, at least some of the data captured by at least some of these sensors can be provided to at least one sensor data manager 316 or other such component or process. The sensor data manager(s) can be responsible for tasks such as formatting or normalizing input sensor data, removing redundant data or reducing noise, removing unreliable data, and so forth. The sensor data manager(s) may accept data from one or more sensors, and then make at least an indicated portion of that data available for analysis. In this example, at least some of the data from the sensor data manager(s) 316 can be provided as input to a user state module 318, component, or process. A user state module 318 can include one or more algorithms, machine learning models, or other such mechanisms to take provided input and attempt to determine, identify, detect, infer, identify, or diagnose a current state, behavior, or other such aspect of a user (or other person or subject) for which sensor data can be obtained. The user state module can be designed to use passive user data, such as data captured by a camera where the user is not required to do anything specific, to monitor a state of the user in a way that is non-intrusive, and in many instances will not even be determinable by a user. Even for sensors that monitor activity, such as keyboard input, the monitoring can be performed in at least some embodiments without the user being required to perform any specific activity or task. The sensor data can capture information about the user, and whatever types of information are available can be used to determine state information. For example, camera image data can be analyzed to determine factors such as heart rate. If additional information is available, then that information can be used to improve the determination or inference. For certain analyses or states, or levels of those states, there may be instances where certain specific actions may be requested of the user as discussed and suggested herein. A user state module may also take in other data, such as information stored to a user repository 334 that is specific to a user. This information may include, for example, known undesired states or behaviors associated with that user, acceptable or unacceptable levels of a given state or behavior, permissions associated with the user as far as which actions can be taken or determinations made, and historical responses of the user when in specific states or exhibiting certain behaviors with respect to specific actions taken.

The user state module 318 can analyze any or all of this and other such information, and can attempt to determine one or more states or behaviors of a user, as well as the levels, extents, and/or types of these states or behaviors. The user state module 318 can then determine whether any of these determinations warrant actions taken to attempt to modify, prevent, or influence one or more states or behaviors (or other such aspects). For example, the module can determine whether the level of a given state exceeds an allowable state threshold, such as an amount of anxiety or depression inferred for the user being above a specified amount. The user state module 318 might also determine more binary factors, such as whether the user is likely experiencing an anxiety attack or PTSD incident, rather than determining a rate, level, or score. As mentioned, the acceptable states, levels, or behaviors may vary for different users, locations, or situations, etc.

If the user state module 318 determines that the state, behaviors, actions, and/or other aspects of the user are all acceptable or within a desired range, for example, then the user state module 318 may not request or instruct any adjustments, and may instead continue monitoring of the sensor data and other data with respect to the user. In some embodiments, a user state module 318 may periodically write information for the user to a data log, such as to establish normal behavior and states of a user under various circumstances, such as to establish an appropriate baseline for the user. If, however, the user state module 318 determines that the user is in an undesired state, or has a probability of being in an undesired state within an upcoming period of time, for example, then the user state module 318 can, in this example, send information about the current state or behavior determination to a state mitigation module 319, or other such component or process. In some embodiments, the user state module 318 might instead use the information itself to determine one or more actions to take. The state mitigation module 319 can use the state or behavior information from the user state module 318 to determine one or more actions to take to attempt to improve, modify, or influence the state or behavior of the user.

In this example, the user state module 318 can work with at least one component manager 320 to attempt to perform one or more actions with respect to the computing device 300. As mentioned, in some embodiments actions may be taken by devices, systems, or components remote from, but in communication with, the computing device 300. This can include, for example, devices connected to a smart home system. The component manager(s) 320 can receive instructions, requests, or calls from the state mitigation module 319, for example, to perform specific actions. The component manager(s) 320 can then provide the appropriate instructions to the relevant components, devices, or processes to perform these actions. For example, the component manager(s) 320 might send an instruction to a display 322 (or display controller) of the computing device to reduce a brightness or contrast of the display. The component manager(s) 320 might send an instruction to a lighting component 324 of the computing device to reduce or modify a color displayed by the device, such as may relate to backlighting of a keyboard, mouse, or computer case. In some embodiments, the backlighting may be caused to display a calming pattern using calming colors, or may slow the rate at which a current pattern is displayed. A border region of a few pixels around a window or display could also be caused to display such a pattern. The component manager(s) 320 might send instructions to a speaker or audio system 326 to reduce or increase volume of playback of audio, as well as potentially instructions to pause, resume, or modify playback. The component manager(s) 320 may send similar instructions to other device settings components 328 or managers to modify operation of the computing device, or cause the computing device to perform specific actions. As mentioned, the component manager(s) 320 might provide instructions to a haptics component 330 or system to provide specific feedback to a user, such as to present the user with a specific haptic pattern as notification of a determined state, or an action to be taken based in part upon a determined state. The component manager(s) 320 might also work with a sensor such as a fingerprint sensor (not illustrated) which can provide data about a Galvanic skin response of the user. For certain user states, such as anxiety or stress, the user may tend to sweat, which can impact the Galvanic skin response measured by the fingerprint sensor. If an air quality sensor is available, data about the blood sugar level of a user can be determined by analyzing the breath of a user. The component manager(s) 320 and/or the state mitigation module 319 can also work with one or more applications, or application managers 332, to modify one or more aspects of specific applications or processes executing on the computing device. This can include, for example, a request for an operating system (OS) to modify a color scheme used or not perform certain types of operations for a period of time, causing an application window to remain hidden or in the background, causing a specific sound or music file to be played, or adjusting a type of content to be displayed, among other such options discussed or suggested herein.

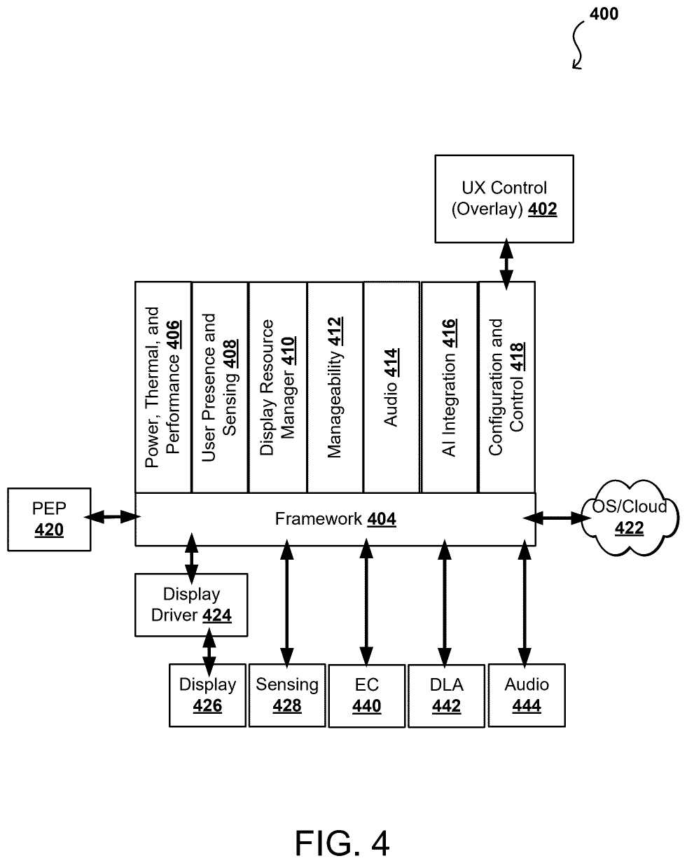

In at least one embodiment, at least some of this functionality may be provided through a framework or platform installed or executing on the computing device. As an example, FIG. 4 illustrates an example computing framework 400 that can be used in accordance with at least one embodiment. In at least one embodiment, various modules or components of this computing framework 400 would be provided via a system-on-chip (SoC) or similar architecture, where at least some of the functionality will be native to the computing device. In at least one embodiment, an entity such as an application developer can make calls to exposed application programming interfaces (APIs) to cause certain functionality to be performed. As illustrated, this example framework 404 can support various supported modules to perform various types of functionality, such as a power, thermal, and performance module 406 for managing these and other such aspects of the device. A user presence and sensing module 408 can accept input from various sensors, such as a camera, sensing component 428, and the like, and can determine information about the user, such as whether the user is looking away from the computing device or no longer near the device, such that the device should operate in a low power state or, after a period of time, power down or perform a similar such actions. Other example modules supported by the framework 404 include a display resource manager module 410, a manageability module 412, an audio module 414, an artificial intelligence (AI) integration module 416, and a configuration and control module 418, among other such options. As illustrated, these modules may also support other module, such as a user interface (UX) control or overlay module 402. In at least one embodiment, the UX control 402 can be provided a third party, such as the provider of the computing device.

As illustrated, the framework can also support various components on, or in communication with, the computing device. This can include interfaces with a display 426 (directly or through a display driver 424), a sensing component 428, an embedded controller (EC) component 440, a deep learning accelerator (DLA) 442, and an audio component 444, among other such options. The framework 404 can facilitate the transfer of instructions to, and feedback or data from, the various hardware components and provide these to the relevant supported modules. The framework 404 can also facilitate the flow of information to/from entities or sources such as a PEP 420 or operating system (OS)/cloud device 422. An application can then use an API of the framework to call functionality of one of the supported modules, which may in turn require interaction with one or more of the hardware components, with the integration provided by the framework 404. In one example use case, data can be gathered from the user presence and sensing module 408 and audio module 414, such as may include information captured about a user, and that information can be fed to the DLA 442 for processing in conjunction with the AI integration module 416 in order to provide information about a state or presence of a user. An anxiety protector may be implemented through the UX control of a third-party provider, which can then leverage the functionality provided via the framework 404.

In at least one embodiment, at least some of the user analysis functionality may be provided through this SoC-based approach, such as where aspects such as pupil size, user heart rate and heart rate variability, emotion/expression, and user respiratory rate can already be calculated using functionality in a module, such as a user presence and sensing module 408, which can leverage data from a camera, microphone, or other such sensor or component of the computing device. Other types of data can be analyzed as discussed elsewhere herein, such as a rate or smoothness of user motion (e.g., as measured through input using a touchscreen, mouse, or keypad), changes in user speech, changes in user motion, and the like. As mentioned, such functionality may also leverage a deep learning accelerator or other such component to provide for high speed, low resource consumption computation using the SoC framework and functionality. As an example, instead of using all GPUs on a graphics card to identify patterns that tend to coincide with an anxiety attack or PTSD, a Deep Learning Accelerator (DLA) can be leveraged to only use a reasonable portion of the GPU power on the card. Once a determination of undesirable state is made, instructions can be provided to a module such as the power, thermal, and performance module 406 and audio module 414 to modify the color scheme or brightness of the display, modify the volume at which audio playback occurs, and so forth.

In at least one embodiment, images captured by at least one camera can be analyzed to determine physical aspects, state, or behaviors of a user, such as by leveraging a framework discussed with respect to FIG. 4. One physical aspect or characteristic is heart rate and/or heart rate variability. Camera-based approaches can utilize a technique such as remote photoplethysmography (PPG) to make measurements based at least in part on image or video data showing a user over a period of time. In such an approach, the image data captured of the user can be analyzed to attempt to determine changes in light absorption over a period of time, as reflected by subtle light variations on the skin, which can be indicative of changes in blood volume over a given cardiac cycle. The changes can be used to determine a current heart rate, and monitoring over time can help to determine the heart rate variability. Each user can have a baseline heart rate value determined, which can be impacted by factor such as age, fitness, and so on. Approaches in accordance with various embodiments can attempt to determine when the heart rate of the user exceeds this baseline by more than an expected or acceptable amount, for example, and can attempt to determine when the variability in heart rate exceeds an acceptable or expected amount. Unexpected fluctuations in heart rate may be indicative of an imbalance in the autonomic nervous system of the person, which can be associated with stress or anxiety.

Similarly, the image data can be analyzed to attempt to determine unexpected or atypical dilation of the pupils of a user. A light sensor of a device can be used to measure the amount of ambient light in a room, and a display driver or other such mechanism can provide a measure of the brightness of a display for a computing device. The user can be monitored over time to set baselines or expected ranges of pupil size for various lighting conditions, such as during daylight outdoors on a sunny day, or at night in a dark room with the display at a low light setting, etc. The image data can be analyzed to isolate or identify the eye regions of the user, then attempt to determine a pupil size (either actual size or relative to another aspect of the user's face, such as relative to the iris diameter or head diameter, etc.). If the pupil appears to be unexpectedly dilated (or smaller in dimension—such as diameter) relative to the expected pupil size or size range for the given lighting conditions, then that unexpected dilation can be reported as dilated pupils are one symptom of stress, where the adrenaline hormones being stimulated to react often result in pupil dilation. An unexpectedly large pupil dimension, on the other hand, may be a sign of high anxiety. As discussed with respect to other sensors or types of data, user baselines and expected ranges can be set, and any unexpected or undesired variation from these baselines or ranges can be reported as being indicative of an undesired user state or behavior. These may include, for example, an unexpected Galvanic skin response measured by a fingerprint scanner, an unexpected rate or pattern of movement determined by analyzing data from a motion sensor, etc. In some embodiments a user may go through a calibration process to establish one or more baselines or expected states or behaviors, but in other embodiments these values or ranges can be learned, such as by further training a machine learning model using this additional user data. As mentioned, the reactions of a user to specific actions can also be learned over time in order to make better decisions as to remedial actions to be taken under specific circumstances for specific types of behaviors or states, etc. In at least one embodiment, there may be default actions applied that may be based in part upon specific aspects or characteristics of a user, as different colors may mean different things or have different impact upon users in different geographic regions or of different cultural backgrounds, etc. As mentioned, a user may also have the ability to set or modify aspects of various options that can be implemented (or prevented from being implemented).

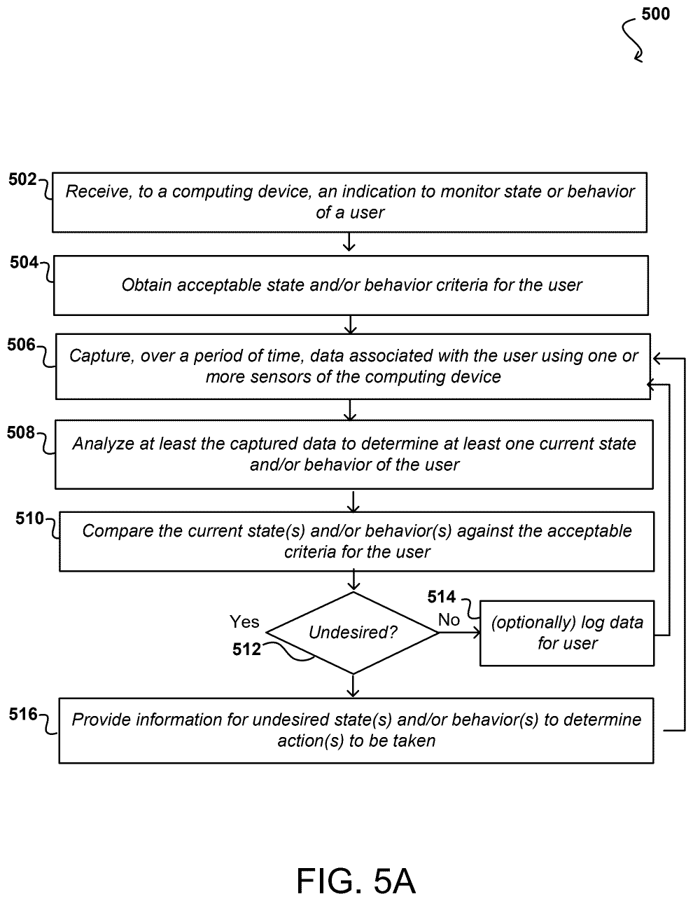

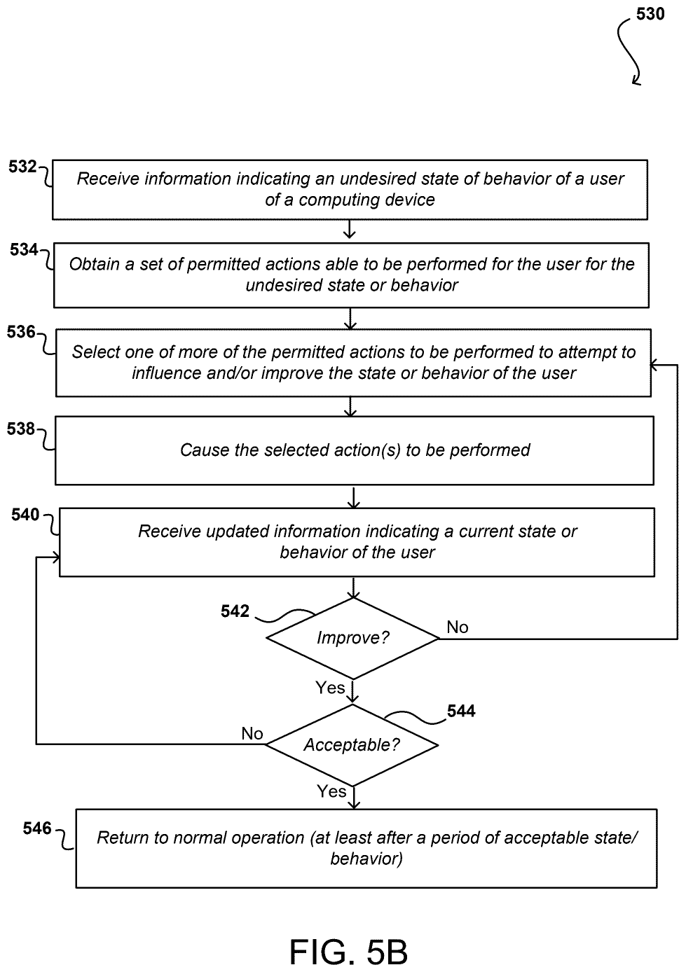

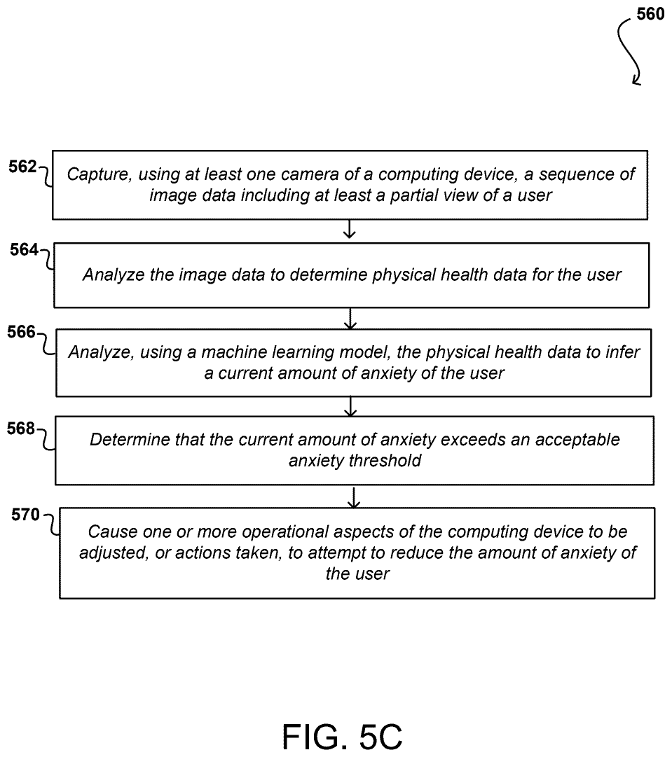

FIG. 5A illustrates an example process 500 for automatically determining a state or a behavior of a person that can be performed in accordance with at least one embodiment. It should be understood that for this and other processes presented herein that there may be additional, fewer, or alternative steps performed or similar or alternative orders, or at least partially in parallel, within the scope of the various embodiments unless otherwise specifically stated. Further, although this example will be discussed with respect to the state or behavior of a user of a computing device, such a process can be used to determine a variety of physical, mental, emotional, or other aspects of a person within a detectable distance of one or more sensors, where data from those sensors is able to be analyzed and one or more actions taken automatically based at least in part thereon. Sensors may be located on different devices, actions to be taken may be performed using different devices, and the analysis and determinations may also be performed on one or more other devices in various embodiments.

In this example, an indication or signal to monitor a state or behavior (or other such aspect) of a user can be received 502 to a computing device (or other device with at least some processing capability). The indication may be provided in response to user input, or provided from a system, application, or operation associated with the user. In some embodiments or instances, the user (or entity authorized to act on behalf of the user) must explicitly provide permission to monitor the user and make determinations or take actions thereon. This permission may be able to be modified over time, or based on locations, environmental circumstances, or other such factors. In addition to the indication, acceptable state and/or behavior criteria can be obtained 504 for the user. This can include, for example, an indication of one or more undesired states of the user, which may correspond to conditions or behaviors which the user is known, believed, or likely to experience. This may include, for example, states associated with anxiety, stress, depression, PTSD, seizures, epilepsy, paranoia, and the like. In some embodiments, this may also include an indication of undesired (or other such) behaviors that may, or may not, be associated with these states. This may include, for example, twitching, pacing, grinding of teeth, shaking, repeating, cursing, or other such behaviors. The criteria may also include levels, state values, categories, or other such factors to use to determine whether one of these states or behaviors of the user rises to an undesired (or other such) level where an action or modification should be taken to attempt to improve, modify, or influence the state or behavior of the user. This may include, for example, a determined level of anxiety or stress. In at least one embodiment, a “level” of stress may be calculated or determined based upon various factors as discussed in more detail elsewhere herein, such as heart rate, heart rate variability, respiratory rate, pupil dilation amount, and the like. In some embodiments, there may be multiple thresholds, such as a first threshold where remedial actions should start to be taken, such as when the user is experiencing a high level of anxiety, and a second threshold where specific, more urgent actions should be taken, such as when the user is experiencing a panic attack, seizure, potentially harmful PTSD episode, and the like. In some embodiments, obtaining acceptable state and/or behavior criteria 504 is performed prior to or concurrently with receiving the indication or signal 502.

In this example, one or more sensors of the computing device can be used to capture 506, over a period of time, data associated with the user, although sensors or data capture devices not directly associated with the computing device can be used as well in at least some embodiments. In at least one embodiment, it can be desired to use sensors to capture passive data that do not require the user to perform a specific task but can instead monitor a state of the user even if the user is not doing anything in particular. As mentioned, the sensor data can include image data, audio data, motion data, and the like. The data can be captured and analyzed over the period of time in order to attempt to determine temporal aspects or behaviors, such as heart rate, heart rate variability, respiratory rate, and the like. At least a portion of this captured data (and any other relevant data) can be analyzed 508 to determine at least one current state and/or behavior of the user. This may include, for example, a current level of anxiety, stress, or depression, among others. The analysis may be performed using one or more algorithms, a machine learning model, or another such approach as discussed and suggested elsewhere herein. The determined state(s) and/or behavior(s) of the user can be compared 510 against the acceptable criteria obtained for the user, such as an acceptable level of anxiety or stress for this user in the current environment. If it is determined 512 that none of the determined states or behaviors satisfy an undesired criterion, then the capturing and analysis of data can continue 506. In at least one embodiment, health or state data for the user can be logged 514, at least periodically, in order to perform tasks such as to update user baselines or identify trends or changes in state or behavior over time. If it is instead determined that at least one state or behavior satisfies at least one undesired criterion, then information for the undesired state and/or behavior can be provided 516 in order to determine, indicate, or signal one or more actions to be taken in response, such as to attempt to improve or influence the state and/or behavior of the user. The capturing and analysis of data continues 506.

FIG. 5B illustrates an example process 530 for determining and performing one or more actions based in part upon a determination of an undesired state or behavior of a user, such as may result from a process as described with respect to FIG. 5A, performed on the same computing device or a different computing device. In this example, information is received 532 indicating an undesired state or behavior of a user of a computing device. This may include not only identification of the state or behavior but also information about the level, value or extent of the state or behavior, as well as potentially data that was used to support the determination. As an example, an indication of a state of high anxiety may include a determined level of current anxiety, as well as information used to calculate that level, as may relate to heart rate, heart rate variability, and so on, which can help to determine which actions to take as well as the extent of those actions. A set of permitted actions can be identified and/or obtained 534 that are able to be performed to address the undesired state of the user. In at least one embodiment, a user can always have the ability to approve or deny any action, or specify a type or amount of that action that can be taken, circumstances in which certain action may be taken or not taken, etc. One or more of the permitted actions can then be selected 536 to be performed to attempt to influence and/or improve the state or behavior of the user. This determination can be made using one or more selection algorithms, a trained machine learning model, or another such approach that can take as input the information about the state or behavior as well as the permitted actions. The actions selected may depend at least in part upon the severity or level of the state or behavior, as well as the actions that are permitted to be taken, as certain combinations of actions may be more or less effective for different levels of severity. The selected action(s) can then be caused 538 to be performed, whether by the computing device or a system, device, component, application, or operation in communication with the computing device and/or associated with the user or a current location of the user. As mentioned, these can include any of a variety of actions or combinations of actions, such as modifying a color scheme, reducing a frequency of notifications, restricting certain content from being presented for a period of time, and the like.

After some period of time, updated information about the current state or behavior of the user can be received 540, which may be received from the same process as the initial indication, such as the process of FIG. 5A. A determination can then be made as to whether the state or behavior has improved since any action was taken. If it is determined 542 that there has been no improvement, at least after a minimal period of time, then an updated set of actions can be selected (if available and permitted) 536 to attempt to improve the state or behavior. This may include selecting at least one different or additional action to be taken, or modifying one of the actions being taken, such as to further restrict content or further reduce a brightness of a display. Although not explicitly illustrated in the figure, if the state or behavior continues to worsen and reaches a dangerous level, then an alarm might be raised or other action taken that might not be available or permitted initially, and depending on the severity or other factors, the monitoring of the user might stop at that point. If it is determined 542 that the state or behavior has improved, then it can also be determined 544 whether the improvement is such that the state or behavior is again within an acceptable range or otherwise again satisfied an acceptable criterion. If it is determined 544 that the state or behavior has improved but still does not satisfy an acceptable criterion, then the process can continue with receiving updated information 540 to monitor the user with the actions being continued that are leading to the improvement. If it is determined 544 that the state and/or behavior of the user satisfies the relevant acceptable criteria, then the computing device (and any related devices, component, systems, processes, etc.) can return 546 to normal or default operation, or at least can stop taking the actions or implementing the modifications that were performed in response to the undesired state or behavior. In at least one embodiment, this return to normal or default operation may only occur after the user has been back in an acceptable state or exhibiting acceptable behavior for at least a minimum period of time, in order to hopefully prevent a relapse or reoccurrence of the state or behavior.

FIG. 5C illustrates an example process 560 that is specific to the detection of high anxiety of a user based on captured image data, which is a specific use case of processes such as those described with respect to FIGS. 5A and 5B. In this example process 560, a sequence of image data (or stream of video data, etc.) is captured 562 using at least one camera of a computing device, where the image data includes at least a partial view of the user (e.g., at least the face of the user). The image data can be analyzed 564 to determine physical health data for the user, such as the current heart rate, heart rate variability, pupil size, or blood pressure of the user. A machine learning model can be used to analyze 566 this physical health data to infer a current amount of anxiety of the user, among other possible states or behaviors. It can be determined 568, algorithmically or otherwise, that this current amount of anxiety exceeds an acceptable anxiety threshold for this user, which may also depend upon the location, time of day, or other such factors. This threshold may be adjustable by the user or another authorized entity. One or more operational aspects of the computing device can then be caused 570 to be adjusted, or actions taken, in order to attempt to reduce or improve the amount of anxiety being exhibited or experienced by the user. In at least some embodiments, these actions or adjustments can be taken by other devices, systems, component, applications, or operations as well. These actions, such as adjusting a brightness, color scheme, music volume, and the like, can attempt to have a calming effect on the user in at least one embodiment, which can help to reduce or improve the level of anxiety in the user. Using such a process, a camera can capture image data of the face of a user over time, and the image data can be analyzed to determine physical aspects of the user such as the current pulse, heart rate variability, and pupil dilation, which are indicative of a level of anxiety of the user. The computing device can then select actions to be taken to attempt to improve the amount of anxiety being experienced by the user while using the computing device. These actions can be taken automatically and discretely in at least some embodiments, such that other people in the area may not be able to determine that adjustments are being made or remedial actions taken. Using only (or primarily) camera-based input without requiring the user to do anything specific also makes such an approach discrete and non-intrusive in many instances.

As mentioned, in at least many embodiments such functionality will be under user control (except where otherwise authorized or permitted, such as control by a parent or guardian). There may be various laws, rules, regulations, or policies that apply to which such an approach must adhere. Further, the user will often want at least some amount of control over when any modification is taken. For example, if a user is in a business meeting in a conference room or is giving a presentation, the user may want to turn off any modifications to avoid embarrassment or potentially making a condition known, particularly in such a setting where the user might be experiencing perspiration or nervousness due to the setting. The user can also activate such functionality when needed, or can provide input such as the user having a stressful day, which may cause thresholds to be lowered or different actions to be taken, among other such options. In some instances, a user may be requested to wear headphones or smart glasses so that certain content can be presented to help influence the state or behavior of the user without such presentation being detectable to others. In other embodiments, a visible prompt or haptic pattern can be presented, and the user must provide some type of confirmatory action before remediation can occur.

As mentioned, monitoring and remediation can occur for various types of “smart” devices able to capture, process, and/or receive data, and perform one or more remedial actions. While notebook, desktop, and tablet computers are provided as primary examples, such devices can also include gaming consoles, smartphones, smart televisions, workstations, and vehicles. For example, a vehicle may already have cameras and sensors that can capture information about a user, and can use a display screen, heads-up display, audio system, or steering wheel haptics to provide calming feedback to a person in the vehicle, as well as dimming the interior lights, adjusting the air flow, modifying a color or pattern of in-vehicle lighting, limiting a number of messages conveyed to the user, and so on. For smart televisions or other components of a smart home, for example, other components or systems in the house may take actions as well, such as to dim the lighting in the room, activate a smart speaker set to play calming music, block notifications from a smart doorbell, and the like.

In at least one embodiment, where permissible and/or approved by the user, the user could also be prompted to perform specific actions. This might include, for example, taking a series of deep breaths or going for a walk. In other instances, the user might be asked to walk through a short exercise on a computing device that should help to improve a current state of the user. The user could be asked to perform meditation or breathing exercises as well. The suggested actions may depend in part upon the location of the user, as a user might be more willing to perform meditation at home in the evening than when at work during business hours. Any or all such actions can be configurable by the user and/or required to have user approval before being suggested, performed, or implemented.

Aspects of various approaches presented herein can be lightweight enough to execute in various locations, such as on a device such as a client device that include a personal computer or gaming console, in real time. For example, data might be captured using sensors of a computing device, that data might be analyzed using machine learning running in the cloud, and then the results used to modify operation of the computing device. Such processing can be performed on, or for, content or information that is generated or captured on, or received by, that client device or received from an external source, such as streaming data or other content received over at least one network from a cloud server 620 or third party service 660, among other such options. In some instances, at least a portion of the processing, generation, compositing, and/or determination of this content may be performed by one of these other devices, systems, or entities, then provided to the client device (or another such recipient) for presentation or another such use.

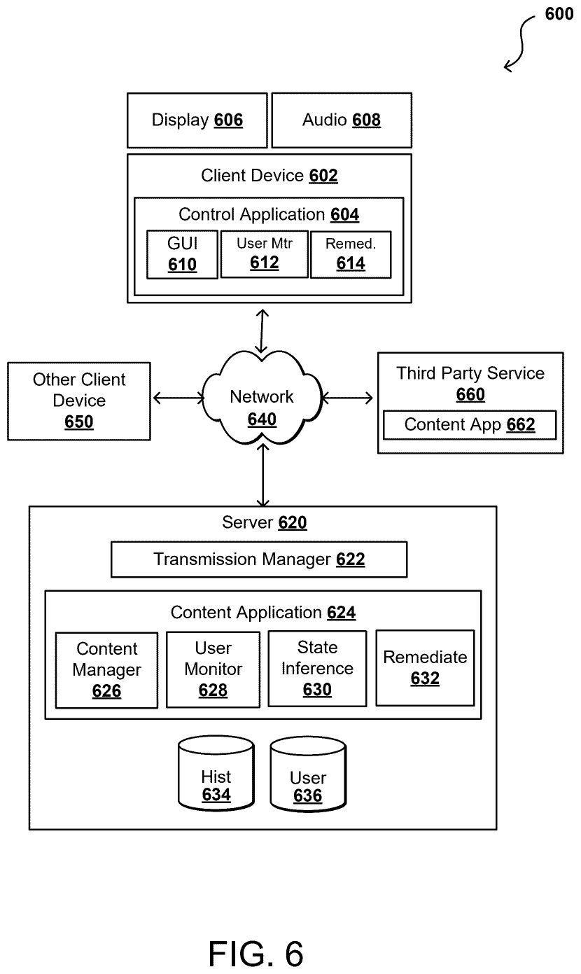

As an example, FIG. 6 illustrates an example network configuration 600 that can be used to provide, generate, modify, encode, process, and/or transmit data, such as captured user data or determined user state data. In at least one embodiment, a client device 602 can generate or receive data for a session using components of a content application 604 on client device 602 and data stored locally on that client device. In at least one embodiment, a content application 624 executing on a server 620 (e.g., a cloud server or edge server) may initiate a session associated with at least one client device 602, as may utilize a session manager and user data stored in a user database 636. A content manager 626 may work with a user monitoring module 628 to determine current user state or behavior, and determine whether any remedial actions should be taken. This can be based at least in part upon expected or acceptable levels of user state or behavior, as may be stored in a historical database 634 or other such repository. The user monitoring module 628 may work with a state inference module 630 to infer a state or behavior of a user, based on captured sensor data or other information, as well as a remediation module 632 if it is determined that the user is in an undesired state or is exhibiting behavior that satisfies at least one remediation criterion, among other such options. In at least one embodiment, the content manager 626 can determine modifications to be made to content to be displayed or operation of the client device 602, or actions to be taken by the client device 602. At least a portion of the determined or modified content or instructions may be transmitted to the client device 602 using an appropriate transmission manager 622 to send by download, streaming, or another such transmission channel. An encoder may be used to encode and/or compress at least some of this data before transmitting to the client device 602. In at least one embodiment, the client device 602 receiving such content can provide this content to a corresponding control application 604, which may also or alternatively include a graphical user interface 610, user monitoring module 612, and remediation module 614 for use in determining the state or behavior of a user of the client device 602, as well as whether to take remedial actions based in part upon the determined state or behavior. A decoder may also be used to decode data received over the network(s) 640 for presentation via client device 602, such as image or video content through a display 606 and audio, such as sounds and music, through at least one audio playback device 608, such as speakers or headphones. In at least one embodiment, at least some of this content may already be stored on, rendered on, or accessible to client device 602 such that transmission over network 640 is not required for at least that portion of content, such as where that content may have been previously downloaded or stored locally on a hard drive or optical disk. In at least one embodiment, a transmission mechanism such as data streaming can be used to transfer this content from server 620, or user database 636, to client device 602. In at least one embodiment, at least a portion of this content can be obtained, enhanced, and/or streamed from another source, such as a third party service 660 or other client device 650, that may also include a content application 662 for generating, enhancing, or providing content. In at least one embodiment, portions of this functionality can be performed using multiple computing devices, or multiple processors within one or more computing devices, such as may include a combination of CPUs and GPUs.

In this example, these client devices can include any appropriate computing devices, as may include a desktop computer, notebook computer, set-top box, streaming device, gaming console, smartphone, tablet computer, VR headset, AR goggles, wearable computer, or a smart television. Each client device can submit a request across at least one wired or wireless network, as may include the Internet, an Ethernet, a local area network (LAN), or a cellular network, among other such options. In this example, these requests can be submitted to an address associated with a cloud provider, who may operate or control one or more electronic resources in a cloud provider environment, such as may include a data center or server farm. In at least one embodiment, the request may be received or processed by at least one edge server, that sits on a network edge and is outside at least one security layer associated with the cloud provider environment. In this way, latency can be reduced by enabling the client devices to interact with servers that are in closer proximity, while also improving security of resources in the cloud provider environment.

In at least one embodiment, such a system can be used for performing graphical rendering operations. In other embodiments, such a system can be used for other purposes, such as for providing image or video content to test or validate autonomous machine applications, or for performing deep learning operations. In at least one embodiment, such a system can be implemented using an edge device, or may incorporate one or more Virtual Machines (VMs). In at least one embodiment, such a system can be implemented at least partially in a data center or at least partially using cloud computing resources.

### Inference and Training Logic

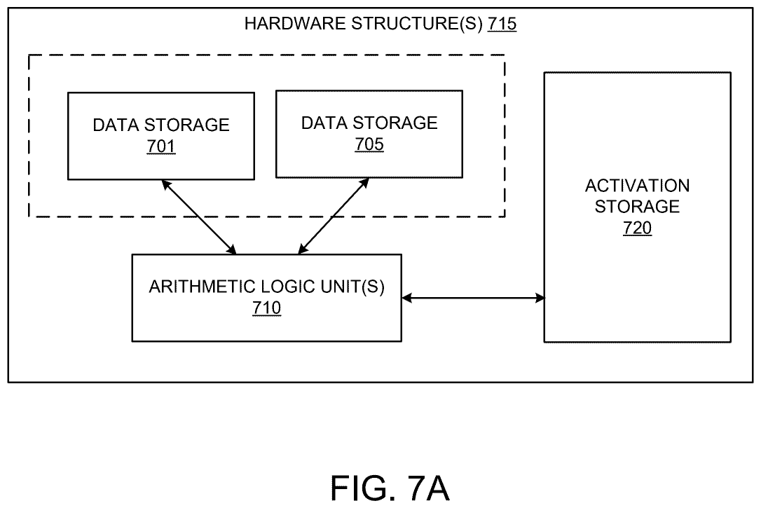

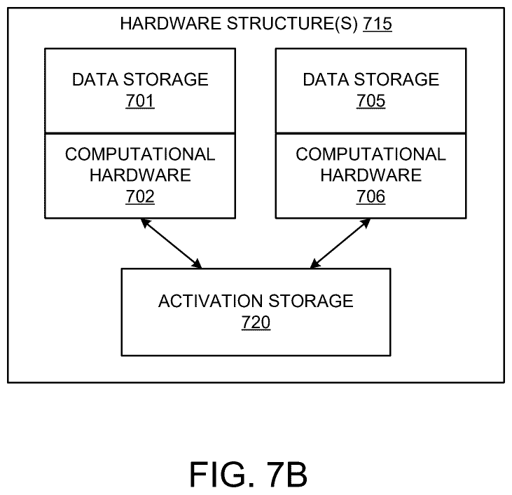

FIG. 7A illustrates inference and/or training logic 715 used to perform inferencing and/or training operations associated with one or more embodiments. Details regarding inference and/or training logic 715 are provided below in conjunction with FIGS. 7A and/or 7B.

In at least one embodiment, inference and/or training logic 715 may include, without limitation, code and/or data storage 701 to store forward and/or output weight and/or input/output data, and/or other parameters to configure neurons or layers of a neural network trained and/or used for inferencing in aspects of one or more embodiments. In at least one embodiment, training logic 715 may include, or be coupled to code and/or data storage 701 to store graph code or other software to control timing and/or order, in which weight and/or other parameter information is to be loaded to configure, logic, including integer and/or floating point units (collectively, arithmetic logic units (ALUs). In at least one embodiment, code, such as graph code, loads weight or other parameter information into processor ALUs based on an architecture of a neural network to which the code corresponds. In at least one embodiment, code and/or data storage 701 stores weight parameters and/or input/output data of each layer of a neural network trained or used in conjunction with one or more embodiments during forward propagation of input/output data and/or weight parameters during training and/or inferencing using aspects of one or more embodiments. In at least one embodiment, any portion of code and/or data storage 701 may be included with other on-chip or off-chip data storage, including a processor's L1, L2, or L3 cache or system memory.

In at least one embodiment, any portion of code and/or data storage 701 may be internal or external to one or more processors or other hardware logic devices or circuits. In at least one embodiment, code and/or data storage 701 may be cache memory, dynamic randomly addressable memory (“DRAM”), static randomly addressable memory (“SRAM”), non-volatile memory (e.g., Flash memory), or other storage. In at least one embodiment, choice of whether code and/or data storage 701 is internal or external to a processor, for example, or comprised of DRAM, SRAM, Flash or some other storage type may depend on available storage on-chip versus off-chip, latency requirements of training and/or inferencing functions being performed, batch size of data used in inferencing and/or training of a neural network, or some combination of these factors.

In at least one embodiment, inference and/or training logic 715 may include, without limitation, a code and/or data storage 705 to store backward and/or output weight and/or input/output data corresponding to neurons or layers of a neural network trained and/or used for inferencing in aspects of one or more embodiments. In at least one embodiment, code and/or data storage 705 stores weight parameters and/or input/output data of each layer of a neural network trained or used in conjunction with one or more embodiments during backward propagation of input/output data and/or weight parameters during training and/or inferencing using aspects of one or more embodiments. In at least one embodiment, training logic 715 may include, or be coupled to code and/or data storage 705 to store graph code or other software to control timing and/or order, in which weight and/or other parameter information is to be loaded to configure, logic, including integer and/or floating point units (collectively, arithmetic logic units (ALUs). In at least one embodiment, code, such as graph code, loads weight or other parameter information into processor ALUs based on an architecture of a neural network to which the code corresponds. In at least one embodiment, any portion of code and/or data storage 705 may be included with other on-chip or off-chip data storage, including a processor's L1, L2, or L3 cache or system memory. In at least one embodiment, any portion of code and/or data storage 705 may be internal or external to on one or more processors or other hardware logic devices or circuits. In at least one embodiment, code and/or data storage 705 may be cache memory, DRAM, SRAM, non-volatile memory (e.g., Flash memory), or other storage. In at least one embodiment, choice of whether code and/or data storage 705 is internal or external to a processor, for example, or comprised of DRAM, SRAM, Flash or some other storage type may depend on available storage on-chip versus off-chip, latency requirements of training and/or inferencing functions being performed, batch size of data used in inferencing and/or training of a neural network, or some combination of these factors.

In at least one embodiment, code and/or data storage 701 and code and/or data storage 705 may be separate storage structures. In at least one embodiment, code and/or data storage 701 and code and/or data storage 705 may be same storage structure. In at least one embodiment, code and/or data storage 701 and code and/or data storage 705 may be partially same storage structure and partially separate storage structures. In at least one embodiment, any portion of code and/or data storage 701 and code and/or data storage 705 may be included with other on-chip or off-chip data storage, including a processor's L1, L2, or L3 cache or system memory.

In at least one embodiment, inference and/or training logic 715 may include, without limitation, one or more arithmetic logic unit(s) (“ALU(s)”) 710, including integer and/or floating point units, to perform logical and/or mathematical operations based, at least in part on, or indicated by, training and/or inference code (e.g., graph code), a result of which may produce activations (e.g., output values from layers or neurons within a neural network) stored in an activation storage 720 that are functions of input/output and/or weight parameter data stored in code and/or data storage 701 and/or code and/or data storage 705. In at least one embodiment, activations stored in activation storage 720 are generated according to linear algebraic and or matrix-based mathematics performed by ALU(s) 710 in response to performing instructions or other code, wherein weight values stored in code and/or data storage 701 and/or code and/or data storage 705 are used as operands along with other values, such as bias values, gradient information, momentum values, or other parameters or hyperparameters, any or all of which may be stored in code and/or data storage 701 or code and/or data storage 705 or another storage on or off-chip.

In at least one embodiment, ALU(s) 710 are included within one or more processors or other hardware logic devices or circuits, whereas in another embodiment, ALU(s) 710 may be external to a processor or other hardware logic device or circuit that uses them (e.g., a co-processor). In at least one embodiment, ALU(s) 710 may be included within a processor's execution units or otherwise within a bank of ALUs accessible by a processor's execution units either within same processor or distributed between different processors of different types (e.g., central processing units, graphics processing units, fixed function units, etc.). In at least one embodiment, code and/or data storage 701, code and/or data storage 705, and activation storage 720 may be on same processor or other hardware logic device or circuit, whereas in another embodiment, they may be in different processors or other hardware logic devices or circuits, or some combination of same and different processors or other hardware logic devices or circuits. In at least one embodiment, any portion of activation storage 720 may be included with other on-chip or off-chip data storage, including a processor's L1, L2, or L3 cache or system memory. Furthermore, inferencing and/or training code may be stored with other code accessible to a processor or other hardware logic or circuit and fetched and/or processed using a processor's fetch, decode, scheduling, execution, retirement and/or other logical circuits.

In at least one embodiment, activation storage 720 may be cache memory, DRAM, SRAM, non-volatile memory (e.g., Flash memory), or other storage. In at least one embodiment, activation storage 720 may be completely or partially within or external to one or more processors or other logical circuits. In at least one embodiment, choice of whether activation storage 720 is internal or external to a processor, for example, or comprised of DRAM, SRAM, Flash or some other storage type may depend on available storage on-chip versus off-chip, latency requirements of training and/or inferencing functions being performed, batch size of data used in inferencing and/or training of a neural network, or some combination of these factors. In at least one embodiment, inference and/or training logic 715 illustrated in FIG. 7A may be used in conjunction with an application-specific integrated circuit (“ASIC”), such as Tensorflow® Processing Unit from Google, an inference processing unit (IPU) from Graphcore™, or a Nervana® (e.g., “Lake Crest”) processor from Intel Corp. In at least one embodiment, inference and/or training logic 715 illustrated in FIG. 7A may be used in conjunction with central processing unit (“CPU”) hardware, graphics processing unit (“GPU”) hardware or other hardware, such as field programmable gate arrays (“FPGAs”).

FIG. 7B illustrates inference and/or training logic 715, according to at least one or more embodiments. In at least one embodiment, inference and/or training logic 715 may include, without limitation, hardware logic in which computational resources are dedicated or otherwise exclusively used in conjunction with weight values or other information corresponding to one or more layers of neurons within a neural network. In at least one embodiment, inference and/or training logic 715 illustrated in FIG. 7B may be used in conjunction with an application-specific integrated circuit (ASIC), such as Tensorflow® Processing Unit from Google, an inference processing unit (IPU) from Graphcore™, or a Nervana® (e.g., “Lake Crest”) processor from Intel Corp. In at least one embodiment, inference and/or training logic 715 illustrated in FIG. 7B may be used in conjunction with central processing unit (CPU) hardware, graphics processing unit (GPU) hardware or other hardware, such as field programmable gate arrays (FPGAs). In at least one embodiment, inference and/or training logic 715 includes, without limitation, code and/or data storage 701 and code and/or data storage 705, which may be used to store code (e.g., graph code), weight values and/or other information, including bias values, gradient information, momentum values, and/or other parameter or hyperparameter information. In at least one embodiment illustrated in FIG. 7B, each of code and/or data storage 701 and code and/or data storage 705 is associated with a dedicated computational resource, such as computational hardware 702 and computational hardware 706, respectively. In at least one embodiment, each of computational hardware 702 and computational hardware 706 comprises one or more ALUs that perform mathematical functions, such as linear algebraic functions, only on information stored in code and/or data storage 701 and code and/or data storage 705, respectively, result of which is stored in activation storage 720.

In at least one embodiment, each of code and/or data storage 701 and 705 and corresponding computational hardware 702 and 706, respectively, correspond to different layers of a neural network, such that resulting activation from one “storage/computational pair 701/702” of code and/or data storage 701 and computational hardware 702 is provided as an input to “storage/computational pair 705/706” of code and/or data storage 705 and computational hardware 706, in order to mirror conceptual organization of a neural network. In at least one embodiment, each of storage/computational pairs 701/702 and 705/706 may correspond to more than one neural network layer. In at least one embodiment, additional storage/computation pairs (not shown) subsequent to or in parallel with storage computation pairs 701/702 and 705/706 may be included in inference and/or training logic 715.

### Data Center

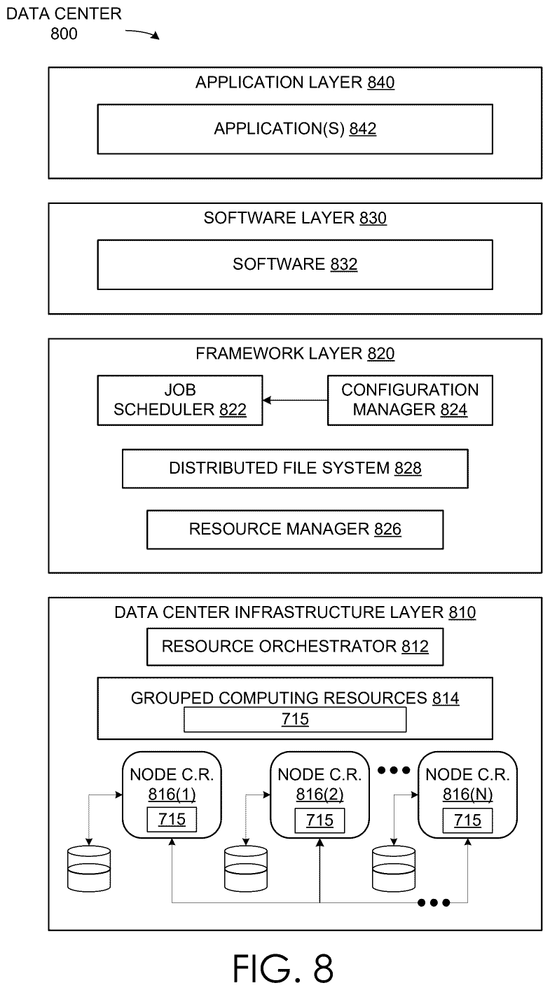

FIG. 8 illustrates an example data center 800, in which at least one embodiment may be used. In at least one embodiment, data center 800 includes a data center infrastructure layer 810, a framework layer 820, a software layer 830, and an application layer 840.

In at least one embodiment, as shown in FIG. 8, data center infrastructure layer 810 may include a resource orchestrator 812, grouped computing resources 814, and node computing resources (“node C.R.s”) 816(1)-816(N), where “N” represents any whole, positive integer. In at least one embodiment, node C.R.s 816(1)-816(N) may include, but are not limited to, any number of central processing units (“CPUs”) or other processors (including accelerators, field programmable gate arrays (FPGAs), graphics processors, etc.), memory devices (e.g., dynamic read-only memory), storage devices (e.g., solid state or disk drives), network input/output (“NW I/O”) devices, network switches, virtual machines (“VMs”), power modules, and cooling modules, etc. In at least one embodiment, one or more node C.R.s from among node C.R.s 816(1)-816(N) may be a server having one or more of above-mentioned computing resources.

In at least one embodiment, grouped computing resources 814 may include separate groupings of node C.R.s housed within one or more racks (not shown), or many racks housed in data centers at various geographical locations (also not shown). Separate groupings of node C.R.s within grouped computing resources 814 may include grouped compute, network, memory or storage resources that may be configured or allocated to support one or more workloads. In at least one embodiment, several node C.R.s including CPUs or processors may grouped within one or more racks to provide compute resources to support one or more workloads. In at least one embodiment, one or more racks may also include any number of power modules, cooling modules, and network switches, in any combination.

In at least one embodiment, resource orchestrator 812 may configure or otherwise control one or more node C.R.s 816(1)-816(N) and/or grouped computing resources 814. In at least one embodiment, resource orchestrator 812 may include a software design infrastructure (“SDI”) management entity for data center 800. In at least one embodiment, resource orchestrator 812 may include hardware, software or some combination thereof.

In at least one embodiment, as shown in FIG. 8, framework layer 820 includes a job scheduler 822, a configuration manager 824, a resource manager 826 and a distributed file system 828. In at least one embodiment, framework layer 820 may include a framework to support software 832 of software layer 830 and/or one or more application(s) 842 of application layer 840. In at least one embodiment, software 832 or application(s) 842 may respectively include web-based service software or applications, such as those provided by Amazon Web Services, Google Cloud and Microsoft Azure. In at least one embodiment, framework layer 820 may be, but is not limited to, a type of free and open-source software web application framework such as Apache Spark™ (hereinafter “Spark”) that may use distributed file system 828 for large-scale data processing (e.g., “big data”). In at least one embodiment, job scheduler 822 may include a Spark driver to facilitate scheduling of workloads supported by various layers of data center 800. In at least one embodiment, configuration manager 824 may be capable of configuring different layers such as software layer 830 and framework layer 820 including Spark and distributed file system 828 for supporting large-scale data processing. In at least one embodiment, resource manager 826 may be capable of managing clustered or grouped computing resources mapped to or allocated for support of distributed file system 828 and job scheduler 822. In at least one embodiment, clustered or grouped computing resources may include grouped computing resource 814 at data center infrastructure layer 810. In at least one embodiment, resource manager 826 may coordinate with resource orchestrator 812 to manage these mapped or allocated computing resources.

In at least one embodiment, software 832 included in software layer 830 may include software used by at least portions of node C.R.s 816(1)-816(N), grouped computing resources 814, and/or distributed file system 828 of framework layer 820. The one or more types of software may include, but are not limited to, Internet web page search software, e-mail virus scan software, database software, and streaming video content software.

In at least one embodiment, application(s) 842 included in application layer 840 may include one or more types of applications used by at least portions of node C.R.s 816(1)-816(N), grouped computing resources 814, and/or distributed file system 828 of framework layer 820. One or more types of applications may include, but are not limited to, any number of a genomics application, a cognitive compute, and a machine learning application, including training or inferencing software, machine learning framework software (e.g., PyTorch, TensorFlow, Caffe, etc.) or other machine learning applications used in conjunction with one or more embodiments.

In at least one embodiment, any of configuration manager 824, resource manager 826, and resource orchestrator 812 may implement any number and type of self-modifying actions based on any amount and type of data acquired in any technically feasible fashion. In at least one embodiment, self-modifying actions may relieve a data center operator of data center 800 from making possibly bad configuration decisions and possibly avoiding underused and/or poor performing portions of a data center.

In at least one embodiment, data center 800 may include tools, services, software or other resources to train one or more machine learning models or predict or infer information using one or more machine learning models according to one or more embodiments described herein. For example, in at least one embodiment, a machine learning model may be trained by calculating weight parameters according to a neural network architecture using software and computing resources described above with respect to data center 800. In at least one embodiment, trained machine learning models corresponding to one or more neural networks may be used to infer or predict information using resources described above with respect to data center 800 by using weight parameters calculated through one or more training techniques described herein.

In at least one embodiment, data center may use CPUs, application-specific integrated circuits (ASICs), GPUs, FPGAs, or other hardware to perform training and/or inferencing using above-described resources. Moreover, one or more software and/or hardware resources described above may be configured as a service to allow users to train or performing inferencing of information, such as image recognition, speech recognition, or other artificial intelligence services.

Inference and/or training logic 715 are used to perform inferencing and/or training operations associated with one or more embodiments. Details regarding inference and/or training logic 715 are provided below in conjunction with FIGS. 7A and/or 7B. In at least one embodiment, inference and/or training logic 715 may be used in system FIG. 8 for inferencing or predicting operations based, at least in part, on weight parameters calculated using neural network training operations, neural network functions and/or architectures, or neural network use cases described herein.

Such components can be used to identify differences between local map data and live perception or observation data, and determine whether to update the local map data for at least the relevant region of a physical environment.

### Computer Systems

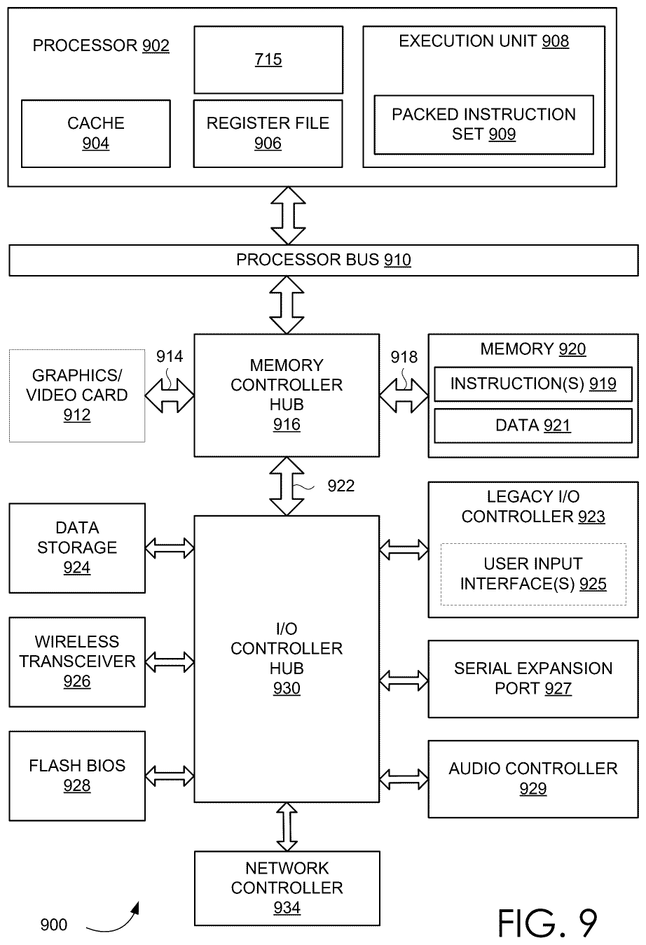

FIG. 9 is a block diagram illustrating an exemplary computer system 900, which may be a system with interconnected devices and components, a system-on-a-chip (SOC) or some combination thereof formed with a processor that may include execution units to execute an instruction, according to at least one embodiment. In at least one embodiment, computer system 900 may include, without limitation, a component, such as a processor 902 to employ execution units including logic to perform algorithms for process data, in accordance with present disclosure, such as in embodiment described herein. In at least one embodiment, computer system 900 may include processors, such as PENTIUM® Processor family, Xeon™, Itanium®, XScale™ and/or StrongARM™, Intel® Core™, or Intel® Nervana™ microprocessors available from Intel Corporation of Santa Clara, California, although other systems (including PCs having other microprocessors, engineering workstations, set-top boxes and like) may also be used. In at least one embodiment, computer system 900 may execute a version of WINDOWS' operating system available from Microsoft Corporation of Redmond, Wash., although other operating systems (UNIX and Linux for example), embedded software, and/or graphical user interfaces, may also be used.

Embodiments may be used in other devices such as handheld devices and embedded applications. Some examples of handheld devices include cellular phones, Internet Protocol devices, digital cameras, personal digital assistants (“PDAs”), and handheld PCs. In at least one embodiment, embedded applications may include a microcontroller, a digital signal processor (“DSP”), system on a chip, network computers (“NetPCs”), set-top boxes, network hubs, wide area network (“WAN”) switches, or any other system that may perform one or more instructions in accordance with at least one embodiment.

In at least one embodiment, computer system 900 may include, without limitation, processor 902 that may include, without limitation, one or more execution unit(s) 908 to perform machine learning model training and/or inferencing according to techniques described herein. In at least one embodiment, computer system 900 is a single processor desktop or server system, but in another embodiment computer system 900 may be a multiprocessor system. In at least one embodiment, processor 902 may include, without limitation, a complex instruction set computing (“CISC”) microprocessor, a reduced instruction set computing (“RISC”) microprocessor, a very long instruction word computing (“VLIW”) microprocessor, a processor implementing a combination of instruction sets, or any other processor device, such as a digital signal processor, for example. In at least one embodiment, processor 902 may be coupled to a processor bus 910 that may transmit data signals between processor 902 and other components in computer system 900.

In at least one embodiment, processor 902 may include, without limitation, a Level 1 (“L1”) internal cache memory (“cache”) 904. In at least one embodiment, processor 902 may have a single internal cache or multiple levels of internal cache. In at least one embodiment, cache 904 may reside external to processor 902. Other embodiments may also include a combination of both internal and external caches depending on particular implementation and needs. In at least one embodiment, register file 906 may store different types of data in various registers including, without limitation, integer registers, floating point registers, status registers, and instruction pointer register.

In at least one embodiment, execution unit(s) 908, including, without limitation, logic to perform integer and floating point operations, also resides in processor 902. In at least one embodiment, processor 902 may also include a microcode (“ucode”) read only memory (“ROM”) that stores microcode for certain macro instructions. In at least one embodiment, execution unit(s) 908 may include logic to handle a packed instruction set 909. In at least one embodiment, by including packed instruction set 909 in an instruction set of a general-purpose processor 902, along with associated circuitry to execute instructions, operations used by many multimedia applications may be performed using packed data in a general-purpose processor 902. In one or more embodiments, many multimedia applications may be accelerated and executed more efficiently by using full width of a processor data bus 910 for performing operations on packed data, which may eliminate need to transfer smaller units of data across processor data bus 910 to perform one or more operations one data element at a time.

In at least one embodiment, execution unit(s) 908 may also be used in microcontrollers, embedded processors, graphics devices, DSPs, and other types of logic circuits. In at least one embodiment, computer system 900 may include, without limitation, a memory 920. In at least one embodiment, memory 920 may be implemented as a Dynamic Random Access Memory (“DRAM”) device, a Static Random Access Memory (“SRAM”) device, flash memory device, or other memory device. In at least one embodiment, memory 920 may store instruction(s) 919 and/or data 921 represented by data signals that may be executed by processor 902.

In at least one embodiment, system logic chip may be coupled to processor bus 910 and memory 920. In at least one embodiment, system logic chip may include, without limitation, a memory controller hub (“MCH”) 916, and processor 902 may communicate with MCH 916 via processor bus 910. In at least one embodiment, MCH 916 may provide a high bandwidth memory path 918 to memory 920 for instruction and data storage and for storage of graphics commands, data and textures. In at least one embodiment, MCH 916 may direct data signals between processor 902, memory 920, and other components in computer system 900 and to bridge data signals between processor bus 910, memory 920, and a system I/O 922. In at least one embodiment, system logic chip may provide a graphics port for coupling to a graphics controller. In at least one embodiment, MCH 916 may be coupled to memory 920 through a high bandwidth memory path 918 and graphics/video card 912 may be coupled to MCH 916 through an Accelerated Graphics Port (“AGP”) interconnect 914.

In at least one embodiment, computer system 900 may use system I/O 922 that is a proprietary hub interface bus to couple MCH 916 to I/O controller hub (“ICH”) 930. In at least one embodiment, ICH 930 may provide direct connections to some I/O devices via a local I/O bus. In at least one embodiment, local I/O bus may include, without limitation, a high-speed I/O bus for connecting peripherals to memory 920, chipset, and processor 902. Examples may include, without limitation, an audio controller 929, a firmware hub (“flash BIOS”) 928, a wireless transceiver 926, a data storage 924, a legacy I/O controller 923 containing user input and keyboard interface(s) 925, a serial expansion port 927, such as Universal Serial Bus (“USB”), and a network controller 934. Data storage 924 may comprise a hard disk drive, a floppy disk drive, a CD-ROM device, a flash memory device, or other mass storage device.

In at least one embodiment, FIG. 9 illustrates a system, which includes interconnected hardware devices or “chips”, whereas in other embodiments, FIG. 9 may illustrate an exemplary System on a Chip (“SoC”). In at least one embodiment, devices may be interconnected with proprietary interconnects, standardized interconnects (e.g., PCIe) or some combination thereof. In at least one embodiment, one or more components of computer system 900 are interconnected using compute express link (CXL) interconnects.

Inference and/or training logic 715 are used to perform inferencing and/or training operations associated with one or more embodiments. Details regarding inference and/or training logic 715 are provided below in conjunction with FIGS. 7A and/or 7B. In at least one embodiment, inference and/or training logic 715 may be used in system FIG. 9 for inferencing or predicting operations based, at least in part, on weight parameters calculated using neural network training operations, neural network functions and/or architectures, or neural network use cases described herein.

Such components can be used to identify differences between local map data and live perception or observation data, and determine whether to update the local map data for at least the relevant region of a physical environment.

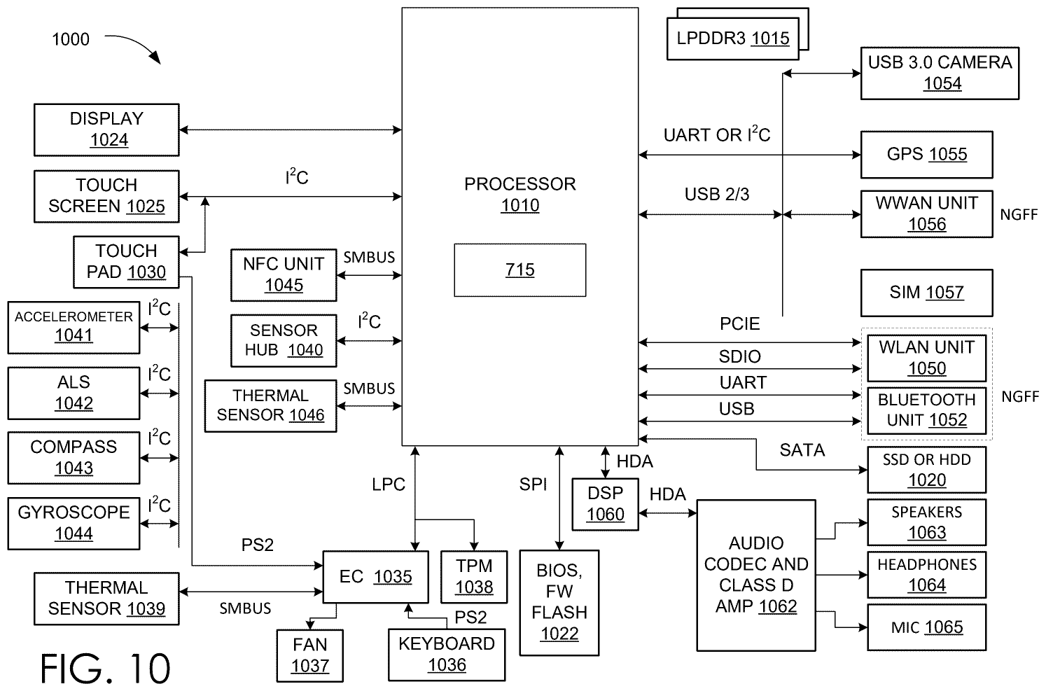

FIG. 10 is a block diagram illustrating an electronic device 1000 for using a processor 1010, according to at least one embodiment. In at least one embodiment, electronic device 1000 may be, for example and without limitation, a notebook, a tower server, a rack server, a blade server, a laptop, a desktop, a tablet, a mobile device, a phone, an embedded computer, or any other suitable electronic device.

In at least one embodiment, electronic device 1000 may include, without limitation, processor 1010 communicatively coupled to any suitable number or kind of components, peripherals, modules, or devices. In at least one embodiment, processor 1010 coupled using a bus or interface, such as a 1° C. bus, a System Management Bus (“SMBus”), a Low Pin Count (LPC) bus, a Serial Peripheral Interface (“SPI”), a High Definition Audio (“HDA”) bus, a Serial Advance Technology Attachment (“SATA”) bus, a Universal Serial Bus (“USB”) (versions 1, 2, 3), or a Universal Asynchronous Receiver/Transmitter (“UART”) bus. In at least one embodiment, FIG. 10 illustrates an electronic device 1000, which includes interconnected hardware devices or “chips”, whereas in other embodiments, FIG. 10 may illustrate an exemplary System on a Chip (“SoC”). In at least one embodiment, devices illustrated in FIG. 10 may be interconnected with proprietary interconnects, standardized interconnects (e.g., PCIe) or some combination thereof. In at least one embodiment, one or more components of FIG. 10 are interconnected using compute express link (CXL) interconnects.

In at least one embodiment, FIG. 10 may include a display 1024, a touch screen 1025, a touch pad 1030, a Near Field Communications unit (“NFC”) 1045, a sensor hub 1040, a thermal sensor 1046, an Express Chipset (“EC”) 1035, a Trusted Platform Module (“TPM”) 1038, BIOS/firmware/flash memory (“BIOS, FW Flash”) 1022, a DSP 1060, a drive 1020 such as a Solid State Disk (“SSD”) or a Hard Disk Drive (“HDD”), a wireless local area network unit (“WLAN”) 1050, a Bluetooth unit 1052, a Wireless Wide Area Network unit (“WWAN”) 1056, a Global Positioning System (GPS) 1055, a camera (“USB 3.0 camera”) 1054 such as a USB 3.0 camera, and/or a Low Power Double Data Rate (“LPDDR”) memory unit (“LPDDR3”) 1015 implemented in, for example, LPDDR3 standard. These components may each be implemented in any suitable manner.

In at least one embodiment, other components may be communicatively coupled to processor 1010 through components discussed above. In at least one embodiment, an accelerometer 1041, Ambient Light Sensor (“ALS”) 1042, compass 1043, and a gyroscope 1044 may be communicatively coupled to sensor hub 1040. In at least one embodiment, thermal sensor 1039, a fan 1037, a keyboard 1036, and a touch pad 1030 may be communicatively coupled to EC 1035. In at least one embodiment, speakers 1063, headphones 1064, and microphone (“mic”) 1065 may be communicatively coupled to an audio unit (“audio codec and class d amp”) 1062, which may in turn be communicatively coupled to DSP 1060. In at least one embodiment, audio unit 1062 may include, for example and without limitation, an audio coder/decoder (“codec”) and a class D amplifier. In at least one embodiment, SIM card (“SIM”) 1057 may be communicatively coupled to WWAN unit 1056. In at least one embodiment, components such as WLAN unit 1050 and Bluetooth unit 1052, as well as WWAN unit 1056 may be implemented in a Next Generation Form Factor (“NGFF”).

Inference and/or training logic 715 are used to perform inferencing and/or training operations associated with one or more embodiments. Details regarding inference and/or training logic 715 are provided below in conjunction with FIGS. 7A and/or 7B. In at least one embodiment, inference and/or training logic 715 may be used in system FIG. 10 for inferencing or predicting operations based, at least in part, on weight parameters calculated using neural network training operations, neural network functions and/or architectures, or neural network use cases described herein.

Such components can be used to identify differences between local map data and live perception or observation data, and determine whether to update the local map data for at least the relevant region of a physical environment.

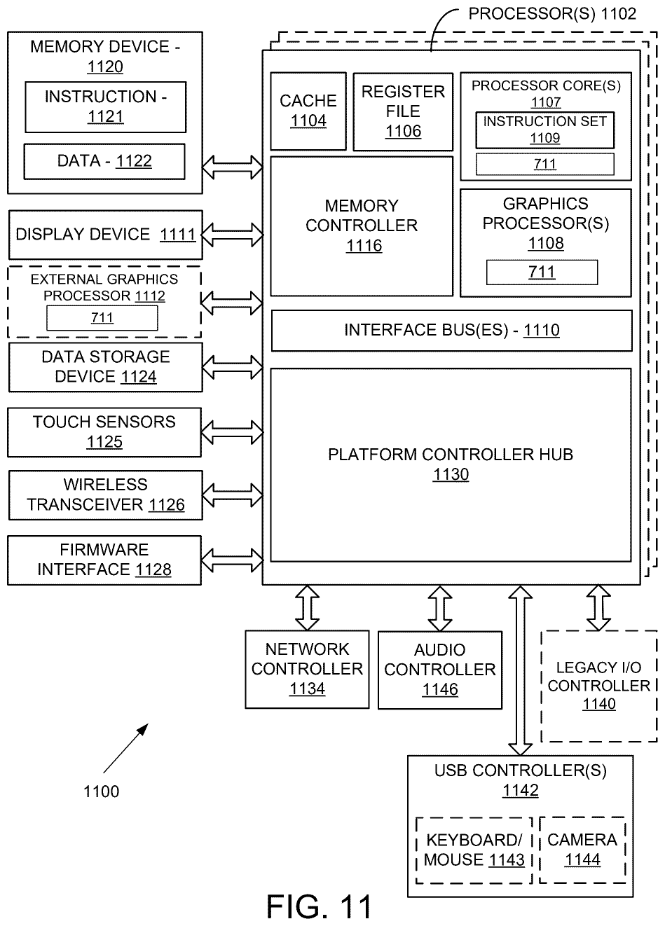

FIG. 11 is a block diagram of a processing system, according to at least one embodiment. In at least one embodiment, processing system 1100 includes one or more processor(s) 1102 and one or more graphics processor(s) 1108, and may be a single processor desktop system, a multiprocessor workstation system, or a server system having a large number of processor(s) 1102 or processor core(s) 1107. In at least one embodiment, processing system 1100 is a processing platform incorporated within a system-on-a-chip (SoC) integrated circuit for use in mobile, handheld, or embedded devices.

In at least one embodiment, processing system 1100 can include, or be incorporated within a server-based gaming platform, a game console, including a game and media console, a mobile gaming console, a handheld game console, or an online game console. In at least one embodiment, processing system 1100 is a mobile phone, smart phone, tablet computing device or mobile Internet device. In at least one embodiment, processing system 1100 can also include, coupled with, or be integrated within a wearable device, such as a smart watch wearable device, smart eyewear device, augmented reality device, or virtual reality device. In at least one embodiment, processing system 1100 is a television or set top box device having one or more processor(s) 1102 and a graphical interface generated by one or more graphics processor(s) 1108.

In at least one embodiment, one or more processor(s) 1102 each include one or more processor core(s) 1107 to process instructions which, when executed, perform operations for system and user software. In at least one embodiment, each of one or more processor core(s) 1107 is configured to process a specific instruction set 1109. In at least one embodiment, instruction set 1109 may facilitate Complex Instruction Set Computing (CISC), Reduced Instruction Set Computing (RISC), or computing via a Very Long Instruction Word (VLIW). In at least one embodiment, processor core(s) 1107 may each process a different instruction set 1109, which may include instructions to facilitate emulation of other instruction sets. In at least one embodiment, processor core(s) 1107 may also include other processing devices, such a Digital Signal Processor (DSP).

In at least one embodiment, processor(s) 1102 includes cache memory (“cache”) 1104. In at least one embodiment, processor(s) 1102 can have a single internal cache or multiple levels of internal cache. In at least one embodiment, cache 1104 is shared among various components of processor(s) 1102. In at least one embodiment, processor(s) 1102 also uses an external cache (e.g., a Level-3 (L3) cache or Last Level Cache (LLC)) (not shown), which may be shared among processor core(s) 1107 using known cache coherency techniques. In at least one embodiment, register file 1106 is additionally included in processor(s) 1102 which may include different types of registers for storing different types of data (e.g., integer registers, floating point registers, status registers, and an instruction pointer register). In at least one embodiment, register file 1106 may include general-purpose registers or other registers.

In at least one embodiment, one or more processor(s) 1102 are coupled with one or more interface bus(es) 1110 to transmit communication signals such as address, data, or control signals between processor(s) 1102 and other components in processing system 1100. In at least one embodiment, interface bus(es) 1110, in one embodiment, can be a processor bus, such as a version of a Direct Media Interface (DMI) bus. In at least one embodiment, interface bus(es) 1110 is not limited to a DMI bus, and may include one or more Peripheral Component Interconnect buses (e.g., PCI, PCI Express), memory buses, or other types of interface buses. In at least one embodiment processor(s) 1102 include an integrated memory controller 1116 and a platform controller hub 1130. In at least one embodiment, memory controller 1116 facilitates communication between a memory device 1120 and other components of processing system 1100, while platform controller hub (PCH) 1130 provides connections to I/O devices via a local I/O bus.

In at least one embodiment, memory device 1120 can be a dynamic random access memory (DRAM) device, a static random access memory (SRAM) device, flash memory device, phase-change memory device, or some other memory device having suitable performance to serve as process memory. In at least one embodiment memory device 1120 can operate as system memory for processing system 1100, to store data 1122 and instruction 1121 for use when one or more processor(s) 1102 executes an application or process. In at least one embodiment, memory controller 1116 also couples with an optional external graphics processor 1112, which may communicate with one or more graphics processor(s) 1108 in processor(s) 1102 to perform graphics and media operations. In at least one embodiment, a display device 1111 can connect to processor(s) 1102. In at least one embodiment display device 1111 can include one or more of an internal display device, as in a mobile electronic device or a laptop device or an external display device attached via a display interface (e.g., DisplayPort, etc.). In at least one embodiment, display device 1111 can include a head mounted display (HMD) such as a stereoscopic display device for use in virtual reality (VR) applications or augmented reality (AR) applications.

In at least one embodiment, platform controller hub 1130 allows peripherals to connect to memory device 1120 and processor(s) 1102 via a high-speed I/O bus. In at least one embodiment, I/O peripherals include, but are not limited to, an audio controller 1146, a network controller 1134, a firmware interface 1128, a wireless transceiver 1126, touch sensors 1125, a data storage device 1124 (e.g., hard disk drive, flash memory, etc.). In at least one embodiment, data storage device 1124 can connect via a storage interface (e.g., SATA) or via a peripheral bus, such as a Peripheral Component Interconnect bus (e.g., PCI, PCI Express). In at least one embodiment, touch sensors 1125 can include touch screen sensors, pressure sensors, or fingerprint sensors. In at least one embodiment, wireless transceiver 1126 can be a Wi-Fi transceiver, a Bluetooth transceiver, or a mobile network transceiver such as a 3G, 4G, or Long Term Evolution (LTE) transceiver. In at least one embodiment, firmware interface 1128 allows communication with system firmware, and can be, for example, a unified extensible firmware interface (UEFI). In at least one embodiment, network controller 1134 can allow a network connection to a wired network. In at least one embodiment, a high-performance network controller (not shown) couples with interface bus(es) 1110. In at least one embodiment, audio controller 1146 is a multi-channel high definition audio controller. In at least one embodiment, processing system 1100 includes an optional legacy I/O controller 1140 for coupling legacy (e.g., Personal System 2 (PS/2)) devices to system. In at least one embodiment, platform controller hub 1130 can also connect to one or more Universal Serial Bus (USB) controller(s) 1142 connect input devices, such as keyboard and mouse 1143 combinations, a camera 1144, or other USB input devices.

In at least one embodiment, an instance of memory controller 1116 and platform controller hub 1130 may be integrated into a discreet external graphics processor, such as external graphics processor 1112. In at least one embodiment, platform controller hub 1130 and/or memory controller 1116 may be external to one or more processor(s) 1102. For example, in at least one embodiment, processing system 1100 can include an external memory controller 1116 and platform controller hub 1130, which may be configured as a memory controller hub and peripheral controller hub within a system chipset that is in communication with processor(s) 1102.

Inference and/or training logic 715 are used to perform inferencing and/or training operations associated with one or more embodiments. Details regarding inference and/or training logic 715 are provided below in conjunction with FIGS. 7A and/or 7B. In at least one embodiment portions or all of inference and/or training logic 715 may be incorporated into processing system 1100. For example, in at least one embodiment, training and/or inferencing techniques described herein may use one or more of ALUs embodied in a graphics processor. Moreover, in at least one embodiment, inferencing and/or training operations described herein may be done using logic other than logic illustrated in FIGS. 7A and/or 7B. In at least one embodiment, weight parameters may be stored in on-chip or off-chip memory and/or registers (shown or not shown) that configure ALUs of a graphics processor to perform one or more machine learning algorithms, neural network architectures, use cases, or training techniques described herein.

Such components can be used to identify differences between local map data and live perception or observation data, and determine whether to update the local map data for at least the relevant region of a physical environment.

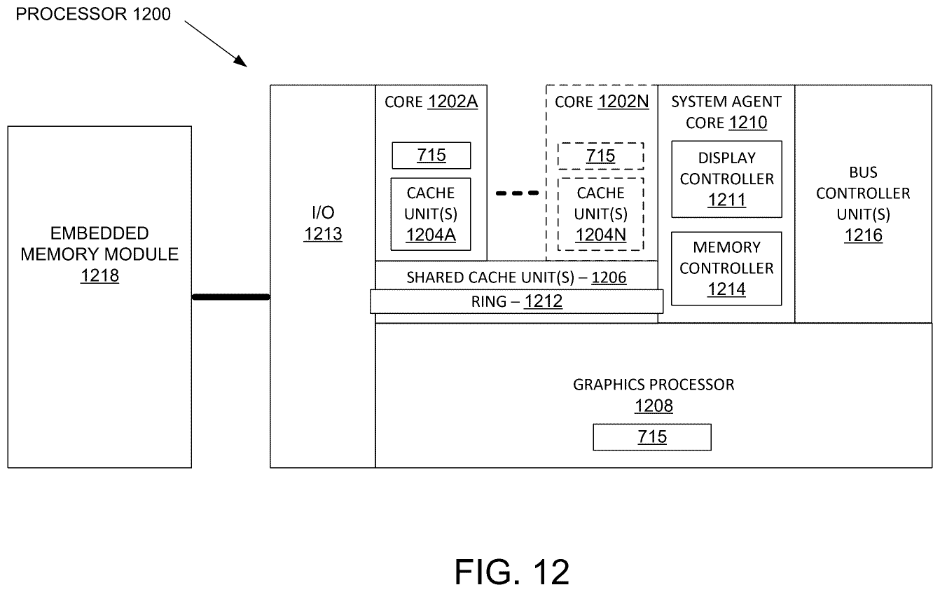

FIG. 12 is a block diagram of a processor 1200 having one or more processor core(s) 1202A-1202N, an integrated memory controller 1214, and an integrated graphics processor 1208, according to at least one embodiment. In at least one embodiment, processor 1200 can include additional cores up to and including additional core(s) 1202N represented by dashed lined boxes. In at least one embodiment, each of processor core(s) 1202A-1202N includes one or more internal cache unit(s) 1204A-1204N. In at least one embodiment, each processor core also has access to one or more shared cached unit(s) 1206.

In at least one embodiment, internal cache unit(s) 1204A-1204N and shared cache unit(s) 1206 represent a cache memory hierarchy within processor 1200. In at least one embodiment, cache memory unit(s) 1204A-1204N may include at least one level of instruction and data cache within each processor core and one or more levels of shared mid-level cache, such as a Level 2 (L2), Level 3 (L3), Level 4 (L4), or other levels of cache, where a highest level of cache before external memory is classified as an LLC. In at least one embodiment, cache coherency logic maintains coherency between various cache unit(s) 1206 and 1204A-1204N.

In at least one embodiment, processor 1200 may also include a set of one or more bus controller unit(s) 1216 and a system agent core 1210. In at least one embodiment, one or more bus controller unit(s) 1216 manage a set of peripheral buses, such as one or more PCI or PCI express buses. In at least one embodiment, system agent core 1210 provides management functionality for various processor components. In at least one embodiment, system agent core 1210 includes one or more integrated memory controller(s) 1214 to manage access to various external memory devices (not shown).

In at least one embodiment, one or more of processor core(s) 1202A-1202N include support for simultaneous multi-threading. In at least one embodiment, system agent core 1210 includes components for coordinating and processor core(s) 1202A-1202N during multi-threaded processing. In at least one embodiment, system agent core 1210 may additionally include a power control unit (PCU), which includes logic and components to regulate one or more power states of processor core(s) 1202A-1202N and graphics processor 1208.

In at least one embodiment, processor 1200 additionally includes graphics processor 1208 to execute graphics processing operations. In at least one embodiment, graphics processor 1208 couples with shared cache unit(s) 1206, and system agent core 1210, including one or more integrated memory controller(s) 1214. In at least one embodiment, system agent core 1210 also includes a display controller 1211 to drive graphics processor output to one or more coupled displays. In at least one embodiment, display controller 1211 may also be a separate module coupled with graphics processor 1208 via at least one interconnect, or may be integrated within graphics processor 1208.

In at least one embodiment, a ring based interconnect unit 1212 is used to couple internal components of processor 1200. In at least one embodiment, an alternative interconnect unit may be used, such as a point-to-point interconnect, a switched interconnect, or other techniques. In at least one embodiment, graphics processor 1208 couples with ring based interconnect unit 1212 via an I/O link 1213.

In at least one embodiment, I/O link 1213 represents at least one of multiple varieties of I/O interconnects, including an on package I/O interconnect which facilitates communication between various processor components and a high-performance embedded memory module 1218, such as an eDRAM module. In at least one embodiment, each of processor core(s) 1202A-1202N and graphics processor 1208 use embedded memory module 1218 as a shared Last Level Cache.

In at least one embodiment, processor core(s) 1202A-1202N are homogenous cores executing a common instruction set architecture. In at least one embodiment, processor core(s) 1202A-1202N are heterogeneous in terms of instruction set architecture (ISA), where one or more of processor core(s) 1202A-1202N execute a common instruction set, while one or more other cores of processor core(s) 1202A-1202N executes a subset of a common instruction set or a different instruction set. In at least one embodiment, processor core(s) 1202A-1202N are heterogeneous in terms of microarchitecture, where one or more cores having a relatively higher power consumption couple with one or more power cores having a lower power consumption. In at least one embodiment, processor 1200 can be implemented on one or more chips or as an SoC integrated circuit.

Inference and/or training logic 715 are used to perform inferencing and/or training operations associated with one or more embodiments. Details regarding inference and/or training logic 715 are provided below in conjunction with FIGS. 7A and/or 7B. In at least one embodiment portions or all of inference and/or training logic 715 may be incorporated into processor 1200. For example, in at least one embodiment, training and/or inferencing techniques described herein may use one or more of ALUs embodied in graphics processor 1208, graphics core(s) 1202A-1202N, or other components in FIG. 12. Moreover, in at least one embodiment, inferencing and/or training operations described herein may be done using logic other than logic illustrated in FIGS. 7A and/or 7B. In at least one embodiment, weight parameters may be stored in on-chip or off-chip memory and/or registers (shown or not shown) that configure ALUs of graphics processor 1200 to perform one or more machine learning algorithms, neural network architectures, use cases, or training techniques described herein.

Such components can be used to identify differences between local map data and live perception or observation data, and determine whether to update the local map data for at least the relevant region of a physical environment.

### Virtualized Computing Platform

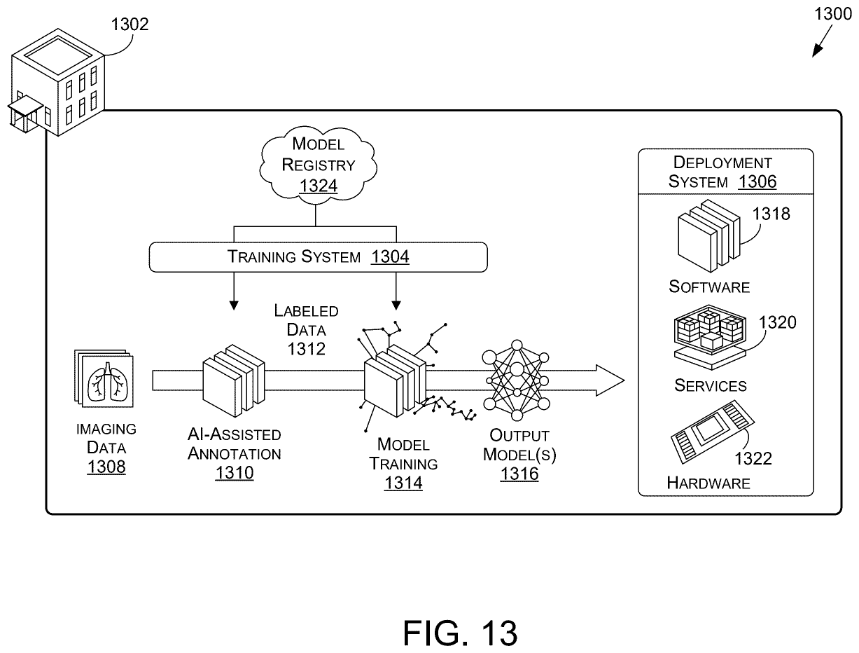

FIG. 13 is an example data flow diagram for a process 1300 of generating and deploying an image processing and inferencing pipeline, in accordance with at least one embodiment. In at least one embodiment, process 1300 may be deployed for use with imaging devices, processing devices, and/or other device types at one or more facility(ies) 1302. Process 1300 may be executed within a training system 1304 and/or a deployment system 1306. In at least one embodiment, training system 1304 may be used to perform training, deployment, and implementation of machine learning models (e.g., neural networks, object detection algorithms, computer vision algorithms, etc.) for use in deployment system 1306. In at least one embodiment, deployment system 1306 may be configured to offload processing and compute resources among a distributed computing environment to reduce infrastructure requirements at facility(ies) 1302. In at least one embodiment, one or more applications in a pipeline may use or call upon services (e.g., inference, visualization, compute, AI, etc.) of deployment system 1306 during execution of applications.

In at least one embodiment, some of applications used in advanced processing and inferencing pipelines may use machine learning models or other AI to perform one or more processing steps. In at least one embodiment, machine learning models may be trained at facility(ies) 1302 using data 1308 (such as imaging data) generated at facility(ies) 1302 (and stored on one or more picture archiving and communication system (PACS) servers at facility(ies) 1302), may be trained using imaging or sequencing data 1308 from another facility(ies), or a combination thereof. In at least one embodiment, training system 1304 may be used to provide applications, services, and/or other resources for generating working, deployable machine learning models for deployment system 1306.

In at least one embodiment, model registry 1324 may be backed by object storage that may support versioning and object metadata. In at least one embodiment, object storage may be accessible through, for example, a cloud storage compatible application programming interface (API) from within a cloud platform. In at least one embodiment, machine learning models within model registry 1324 may uploaded, listed, modified, or deleted by developers or partners of a system interacting with an API. In at least one embodiment, an API may provide access to methods that allow users with appropriate credentials to associate models with applications, such that models may be executed as part of execution of containerized instantiations of applications.

In at least one embodiment, training pipeline 1304 (FIG. 13) may include a scenario where facility(ies) 1302 is training their own machine learning model, or has an existing machine learning model that needs to be optimized or updated. In at least one embodiment, imaging data 1308 generated by imaging device(s), sequencing devices, and/or other device types may be received. In at least one embodiment, once imaging data 1308 is received, AI-assisted annotation 1310 may be used to aid in generating annotations corresponding to imaging data 1308 to be used as ground truth data for a machine learning model. In at least one embodiment, AI-assisted annotation 1310 may include one or more machine learning models (e.g., convolutional neural networks (CNNs)) that may be trained to generate annotations corresponding to certain types of imaging data 1308 (e.g., from certain devices). In at least one embodiment, AI-assisted annotation 1310 may then be used directly, or may be adjusted or fine-tuned using an annotation tool to generate ground truth data. In at least one embodiment, AI-assisted annotation 1310, labeled data 1312, or a combination thereof may be used as ground truth data for training a machine learning model. In at least one embodiment, a trained machine learning model may be referred to as output model(s) 1316, and may be used by deployment system 1306, as described herein.

In at least one embodiment, a training pipeline may include a scenario where facility(ies) 1302 needs a machine learning model for use in performing one or more processing tasks for one or more applications in deployment system 1306, but facility(ies) 1302 may not currently have such a machine learning model (or may not have a model that is optimized, efficient, or effective for such purposes). In at least one embodiment, an existing machine learning model may be selected from a model registry 1324. In at least one embodiment, model registry 1324 may include machine learning models trained to perform a variety of different inference tasks on imaging data. In at least one embodiment, machine learning models in model registry 1324 may have been trained on imaging data from different facilities than facility(ies) 1302 (e.g., facilities remotely located). In at least one embodiment, machine learning models may have been trained on imaging data from one location, two locations, or any number of locations. In at least one embodiment, when being trained on imaging data from a specific location, training may take place at that location, or at least in a manner that protects confidentiality of imaging data or restricts imaging data from being transferred off-premises. In at least one embodiment, once a model is trained—or partially trained—at one location, a machine learning model may be added to model registry 1324. In at least one embodiment, a machine learning model may then be retrained, or updated, at any number of other facilities, and a retrained or updated model may be made available in model registry 1324. In at least one embodiment, a machine learning model may then be selected from model registry 1324—and referred to as output model(s) 1316—and may be used in deployment system 1306 to perform one or more processing tasks for one or more applications of a deployment system.

In at least one embodiment, a scenario may include facility(ies) 1302 requiring a machine learning model for use in performing one or more processing tasks for one or more applications in deployment system 1306, but facility(ies) 1302 may not currently have such a machine learning model (or may not have a model that is optimized, efficient, or effective for such purposes). In at least one embodiment, a machine learning model selected from model registry 1324 may not be fine-tuned or optimized for imaging data 1308 generated at facility(ies) 1302 because of differences in populations, robustness of training data used to train a machine learning model, diversity in anomalies of training data, and/or other issues with training data. In at least one embodiment, AI-assisted annotation 1310 may be used to aid in generating annotations corresponding to imaging data 1308 to be used as ground truth data for retraining or updating a machine learning model. In at least one embodiment, labeled data 1312 may be used as ground truth data for training a machine learning model. In at least one embodiment, retraining or updating a machine learning model may be referred to as model training 1314. In at least one embodiment, model training 1314—e.g., AI-assisted annotation 1310, labeled data 1312, or a combination thereof—may be used as ground truth data for retraining or updating a machine learning model. In at least one embodiment, a trained machine learning model may be referred to as output model(s) 1316, and may be used by deployment system 1306, as described herein.

In at least one embodiment, deployment system 1306 may include software 1318, services 1320, hardware 1322, and/or other components, features, and functionality. In at least one embodiment, deployment system 1306 may include a software “stack,” such that software 1318 may be built on top of services 1320 and may use services 1320 to perform some or all of processing tasks, and services 1320 and software 1318 may be built on top of hardware 1322 and use hardware 1322 to execute processing, storage, and/or other compute tasks of deployment system 1306. In at least one embodiment, software 1318 may include any number of different containers, where each container may execute an instantiation of an application. In at least one embodiment, each application may perform one or more processing tasks in an advanced processing and inferencing pipeline (e.g., inferencing, object detection, feature detection, segmentation, image enhancement, calibration, etc.). In at least one embodiment, an advanced processing and inferencing pipeline may be defined based on selections of different containers that are desired or required for processing imaging data 1308, in addition to containers that receive and configure imaging data for use by each container and/or for use by facility(ies) 1302 after processing through a pipeline (e.g., to convert outputs back to a usable data type). In at least one embodiment, a combination of containers within software 1318 (e.g., that make up a pipeline) may be referred to as a virtual instrument (as described in more detail herein), and a virtual instrument may leverage services 1320 and hardware 1322 to execute some or all processing tasks of applications instantiated in containers.

In at least one embodiment, a data processing pipeline may receive input data (e.g., imaging data 1308) in a specific format in response to an inference request (e.g., a request from a user of deployment system 1306). In at least one embodiment, input data may be representative of one or more images, video, and/or other data representations generated by one or more imaging devices. In at least one embodiment, data may undergo pre-processing as part of data processing pipeline to prepare data for processing by one or more applications. In at least one embodiment, post-processing may be performed on an output of one or more inferencing tasks or other processing tasks of a pipeline to prepare an output data for a next application and/or to prepare output data for transmission and/or use by a user (e.g., as a response to an inference request). In at least one embodiment, inferencing tasks may be performed by one or more machine learning models, such as trained or deployed neural networks, which may include output model(s) 1316 of training system 1304.

In at least one embodiment, tasks of data processing pipeline may be encapsulated in a container(s) that each represents a discrete, fully functional instantiation of an application and virtualized computing environment that is able to reference machine learning models. In at least one embodiment, containers or applications may be published into a private (e.g., limited access) area of a container registry (described in more detail herein), and trained or deployed models may be stored in model registry 1324 and associated with one or more applications. In at least one embodiment, images of applications (e.g., container images) may be available in a container registry, and once selected by a user from a container registry for deployment in a pipeline, an image may be used to generate a container for an instantiation of an application for use by a user's system.

In at least one embodiment, developers (e.g., software developers, clinicians, doctors, etc.) may develop, publish, and store applications (e.g., as containers) for performing image processing and/or inferencing on supplied data. In at least one embodiment, development, publishing, and/or storing may be performed using a software development kit (SDK) associated with a system (e.g., to ensure that an application and/or container developed is compliant with or compatible with a system). In at least one embodiment, an application that is developed may be tested locally (e.g., at a first facility, on data from a first facility) with an SDK which may support at least some of services 1320 as a system (e.g., processor 1200 of FIG. 12). In at least one embodiment, because DICOM objects may contain anywhere from one to hundreds of images or other data types, and due to a variation in data, a developer may be responsible for managing (e.g., setting constructs for, building pre-processing into an application, etc.) extraction and preparation of incoming data. In at least one embodiment, once validated by process 1300 (e.g., for accuracy), an application may be available in a container registry for selection and/or implementation by a user to perform one or more processing tasks with respect to data at a facility (e.g., a second facility) of a user.

In at least one embodiment, developers may then share applications or containers through a network for access and use by users of a system (e.g., process 1300 of FIG. 13). In at least one embodiment, completed and validated applications or containers may be stored in a container registry and associated machine learning models may be stored in model registry 1324. In at least one embodiment, a requesting entity-who provides an inference or image processing request—may browse a container registry and/or model registry 1324 for an application, container, dataset, machine learning model, etc., select a desired combination of elements for inclusion in data processing pipeline, and submit an imaging processing request. In at least one embodiment, a request may include input data (and associated patient data, in some examples) that is necessary to perform a request, and/or may include a selection of application(s) and/or machine learning models to be executed in processing a request. In at least one embodiment, a request may then be passed to one or more components of deployment system 1306 (e.g., a cloud) to perform processing of data processing pipeline. In at least one embodiment, processing by deployment system 1306 may include referencing selected elements (e.g., applications, containers, models, etc.) from a container registry and/or model registry 1324. In at least one embodiment, once results are generated by a pipeline, results may be returned to a user for reference (e.g., for viewing in a viewing application suite executing on a local, on-premises workstation or terminal).

In at least one embodiment, to aid in processing or execution of applications or containers in pipelines, services 1320 may be leveraged. In at least one embodiment, services 1320 may include compute services, artificial intelligence (AI) services, visualization services, and/or other service types. In at least one embodiment, services 1320 may provide functionality that is common to one or more applications in software 1318, so functionality may be abstracted to a service that may be called upon or leveraged by applications. In at least one embodiment, functionality provided by services 1320 may run dynamically and more efficiently, while also scaling well by allowing applications to process data in parallel (e.g., using a parallel computing platform). In at least one embodiment, rather than each application that shares a same functionality offered by services 1320 being required to have a respective instance of services 1320, services 1320 may be shared between and among various applications. In at least one embodiment, services 1320 may include an inference server or engine that may be used for executing detection or segmentation tasks, as non-limiting examples. In at least one embodiment, a model training service may be included that may provide machine learning model training and/or retraining capabilities. In at least one embodiment, a data augmentation service may further be included that may provide GPU accelerated data (e.g., DICOM, RIS, CIS, REST compliant, RPC, raw, etc.) extraction, resizing, scaling, and/or other augmentation. In at least one embodiment, a visualization service may be used that may add image rendering effects—such as ray-tracing, rasterization, denoising, sharpening, etc.—to add realism to two-dimensional (2D) and/or three-dimensional (3D) models. In at least one embodiment, virtual instrument services may be included that provide for beam-forming, segmentation, inferencing, imaging, and/or support for other applications within pipelines of virtual instruments.

In at least one embodiment, where a services 1320 includes an AI service (e.g., an inference service), one or more machine learning models may be executed by calling upon (e.g., as an API call) an inference service (e.g., an inference server) to execute machine learning model(s), or processing thereof, as part of application execution. In at least one embodiment, where another application includes one or more machine learning models for segmentation tasks, an application may call upon an inference service to execute machine learning models for performing one or more of processing operations associated with segmentation tasks. In at least one embodiment, software 1318 implementing advanced processing and inferencing pipeline that includes segmentation application and anomaly detection application may be streamlined because each application may call upon a same inference service to perform one or more inferencing tasks.

In at least one embodiment, hardware 1322 may include GPUs, CPUs, graphics cards, an AI/deep learning system (e.g., an AI supercomputer, such as NVIDIA's DGX), a cloud platform, or a combination thereof. In at least one embodiment, different types of hardware 1322 may be used to provide efficient, purpose-built support for software 1318 and services 1320 in deployment system 1306. In at least one embodiment, use of GPU processing may be implemented for processing locally (e.g., at facility(ies) 1302), within an AI/deep learning system, in a cloud system, and/or in other processing components of deployment system 1306 to improve efficiency, accuracy, and efficacy of image processing and generation. In at least one embodiment, software 1318 and/or services 1320 may be optimized for GPU processing with respect to deep learning, machine learning, and/or high-performance computing, as non-limiting examples. In at least one embodiment, at least some of computing environment of deployment system 1306 and/or training system 1304 may be executed in a datacenter one or more supercomputers or high performance computing systems, with GPU optimized software (e.g., hardware and software combination of NVIDIA's DGX System). In at least one embodiment, hardware 1322 may include any number of GPUs that may be called upon to perform processing of data in parallel, as described herein. In at least one embodiment, cloud platform may further include GPU processing for GPU-optimized execution of deep learning tasks, machine learning tasks, or other computing tasks. In at least one embodiment, cloud platform (e.g., NVIDIA's NGC) may be executed using an AI/deep learning supercomputer(s) and/or GPU-optimized software (e.g., as provided on NVIDIA's DGX Systems) as a hardware abstraction and scaling platform. In at least one embodiment, cloud platform may integrate an application container clustering system or orchestration system (e.g., KUBERNETES) on multiple GPUs to allow seamless scaling and load balancing.

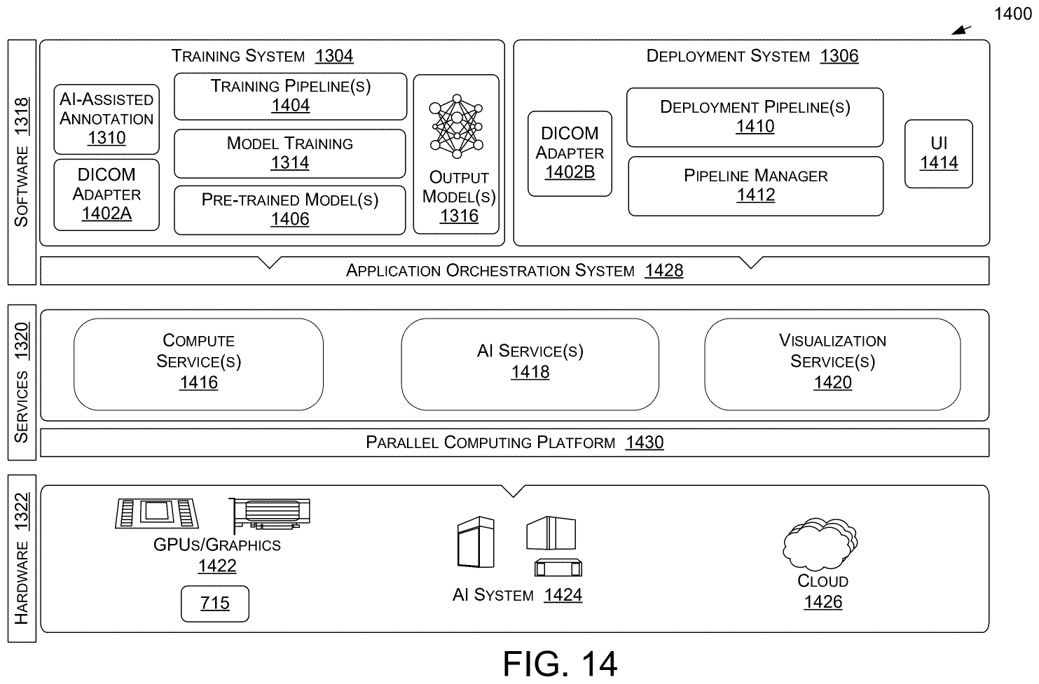

FIG. 14 is a system diagram for an example system 1400 for generating and deploying an imaging deployment pipeline, in accordance with at least one embodiment. In at least one embodiment, system 1400 may be used to implement process 1300 of FIG. 13 and/or other processes including advanced processing and inferencing pipelines. In at least one embodiment, system 1400 may include training system 1304 and deployment system 1306. In at least one embodiment, training system 1304 and deployment system 1306 may be implemented using software 1318, services 1320, and/or hardware 1322, as described herein.

In at least one embodiment, system 1400 (e.g., training system 1304 and/or deployment system 1306) may implemented in a cloud computing environment (e.g., using cloud 1426). In at least one embodiment, system 1400 may be implemented locally with respect to a healthcare services facility, or as a combination of both cloud and local computing resources. In at least one embodiment, access to APIs in cloud 1426 may be restricted to authorized users through enacted security measures or protocols. In at least one embodiment, a security protocol may include web tokens that may be signed by an authentication (e.g., AuthN, AuthZ, Gluecon, etc.) service and may carry appropriate authorization. In at least one embodiment, APIs of virtual instruments (described herein), or other instantiations of system 1400, may be restricted to a set of public IPs that have been vetted or authorized for interaction.

In at least one embodiment, various components of system 1400 may communicate between and among one another using any of a variety of different network types, including but not limited to local area networks (LANs) and/or wide area networks (WANs) via wired and/or wireless communication protocols. In at least one embodiment, communication between facilities and components of system 1400 (e.g., for transmitting inference requests, for receiving results of inference requests, etc.) may be communicated over data bus(ses), wireless data protocols (Wi-Fi), wired data protocols (e.g., Ethernet), etc.

In at least one embodiment, training system 1304 may execute training pipeline(s) 1404, similar to those described herein with respect to FIG. 13. In at least one embodiment, where one or more machine learning models are to be used in deployment pipeline(s) 1410 by deployment system 1306, training pipeline(s) 1404 may be used to train or retrain one or more (e.g. pre-trained) models, and/or implement one or more of pre-trained model(s) 1406 (e.g., without a need for retraining or updating). In at least one embodiment, as a result of training pipeline(s) 1404, output model(s) 1316 may be generated. In at least one embodiment, training pipeline(s) 1404 may include any number of processing steps, such as but not limited to imaging data (or other input data) conversion or adaption In at least one embodiment, for different machine learning models used by deployment system 1306, different training pipeline(s) 1404 may be used. In at least one embodiment, training pipeline(s) 1404 similar to a first example described with respect to FIG. 13 may be used for a first machine learning model, training pipeline(s) 1404 similar to a second example described with respect to FIG. 13 may be used for a second machine learning model, and training pipeline(s) 1404 similar to a third example described with respect to FIG. 13 may be used for a third machine learning model. In at least one embodiment, any combination of tasks within training system 1304 may be used depending on what is required for each respective machine learning model. In at least one embodiment, one or more of machine learning models may already be trained and ready for deployment so machine learning models may not undergo any processing by training system 1304, and may be implemented by deployment system 1306.

In at least one embodiment, output model(s) 1316 and/or pre-trained model(s) 1406 may include any types of machine learning models depending on implementation or embodiment. In at least one embodiment, and without limitation, machine learning models used by system 1400 may include machine learning model(s) using linear regression, logistic regression, decision trees, support vector machines (SVM), Naïve Bayes, k-nearest neighbor (Knn), K means clustering, random forest, dimensionality reduction algorithms, gradient boosting algorithms, neural networks (e.g., auto-encoders, convolutional, recurrent, perceptrons, Long/Short Term Memory (LSTM), Hopfield, Boltzmann, deep belief, deconvolutional, generative adversarial, liquid state machine, etc.), and/or other types of machine learning models.

In at least one embodiment, training pipeline(s) 1404 may include AI-assisted annotation, as described in more detail herein with respect to at least FIG. 14. In at least one embodiment, labeled data 1312 (e.g., traditional annotation) may be generated by any number of techniques. In at least one embodiment, labels or other annotations may be generated within a drawing program (e.g., an annotation program), a computer aided design (CAD) program, a labeling program, another type of program suitable for generating annotations or labels for ground truth, and/or may be hand drawn, in some examples. In at least one embodiment, ground truth data may be synthetically produced (e.g., generated from computer models or renderings), real produced (e.g., designed and produced from real-world data), machine-automated (e.g., using feature analysis and learning to extract features from data and then generate labels), human annotated (e.g., labeler, or annotation expert, defines location of labels), and/or a combination thereof. In at least one embodiment, for each instance of imaging data 1308 (or other data type used by machine learning models), there may be corresponding ground truth data generated by training system 1304. In at least one embodiment, AI-assisted annotation 1310 may be performed as part of deployment pipelines 1410; either in addition to, or in lieu of AI-assisted annotation 1310 included in training pipeline(s) 1404. In at least one embodiment, system 1400 may include a multi-layer platform that may include a software layer (e.g., software 1318) of diagnostic applications (or other application types) that may perform one or more medical imaging and diagnostic functions. In at least one embodiment, system 1400 may be communicatively coupled to (e.g., via encrypted links) PACS server networks of one or more facilities. In at least one embodiment, system 1400 may be configured to access and referenced data from PACS servers to perform operations, such as training machine learning models, deploying machine learning models, image processing, inferencing, and/or other operations.

In at least one embodiment, a software layer may be implemented as a secure, encrypted, and/or authenticated API through which applications or containers may be invoked (e.g., called) from an external environment(s) (e.g., facility(ies) 1302). In at least one embodiment, applications may then call or execute one or more services 1320 for performing compute, AI, or visualization tasks associated with respective applications, and software 1318 and/or services 1320 may leverage hardware 1322 to perform processing tasks in an effective and efficient manner. In at least one embodiment, communications sent to, or received by, a training system 1304 and a deployment system 1306 may occur using a pair of DICOM adapters 1402A, 1402B.

In at least one embodiment, deployment system 1306 may execute deployment pipeline(s) 1410. In at least one embodiment, deployment pipeline(s) 1410 may include any number of applications that may be sequentially, non-sequentially, or otherwise applied to imaging data (and/or other data types) generated by imaging devices, sequencing devices, genomics devices, etc.—including AI-assisted annotation, as described above. In at least one embodiment, as described herein, a deployment pipeline(s) 1410 for an individual device may be referred to as a virtual instrument for a device (e.g., a virtual ultrasound instrument, a virtual CT scan instrument, a virtual sequencing instrument, etc.). In at least one embodiment, for a single device, there may be more than one deployment pipeline(s) 1410 depending on information desired from data generated by a device. In at least one embodiment, where detections of anomalies are desired from an MRI machine, there may be a first deployment pipeline(s) 1410, and where image enhancement is desired from output of an MRI machine, there may be a second deployment pipeline(s) 1410.

In at least one embodiment, an image generation application may include a processing task that includes use of a machine learning model. In at least one embodiment, a user may desire to use their own machine learning model, or to select a machine learning model from model registry 1324. In at least one embodiment, a user may implement their own machine learning model or select a machine learning model for inclusion in an application for performing a processing task. In at least one embodiment, applications may be selectable and customizable, and by defining constructs of applications, deployment and implementation of applications for a particular user are presented as a more seamless user experience. In at least one embodiment, by leveraging other features of system 1400—such as services 1320 and hardware 1322—deployment pipeline(s) 1410 may be even more user friendly, provide for easier integration, and produce more accurate, efficient, and timely results.

In at least one embodiment, deployment system 1306 may include a user interface (“UI”) 1414 (e.g., a graphical user interface, a web interface, etc.) that may be used to select applications for inclusion in deployment pipeline(s) 1410, arrange applications, modify or change applications or parameters or constructs thereof, use and interact with deployment pipeline(s) 1410 during set-up and/or deployment, and/or to otherwise interact with deployment system 1306. In at least one embodiment, although not illustrated with respect to training system 1304, UI 1414 (or a different user interface) may be used for selecting models for use in deployment system 1306, for selecting models for training, or retraining, in training system 1304, and/or for otherwise interacting with training system 1304.

In at least one embodiment, pipeline manager 1412 may be used, in addition to an application orchestration system 1428, to manage interaction between applications or containers of deployment pipeline(s) 1410 and services 1320 and/or hardware 1322. In at least one embodiment, pipeline manager 1412 may be configured to facilitate interactions from application to application, from application to services 1320, and/or from application or service to hardware 1322. In at least one embodiment, although illustrated as included in software 1318, this is not intended to be limiting, and in some examples pipeline manager 1412 may be included in services 1320. In at least one embodiment, application orchestration system 1428 (e.g., Kubernetes, DOCKER, etc.) may include a container orchestration system that may group applications into containers as logical units for coordination, management, scaling, and deployment. In at least one embodiment, by associating applications from deployment pipeline(s) 1410 (e.g., a reconstruction application, a segmentation application, etc.) with individual containers, each application may execute in a self-contained environment (e.g., at a kernel level) to increase speed and efficiency.

In at least one embodiment, each application and/or container (or image thereof) may be individually developed, modified, and deployed (e.g., a first user or developer may develop, modify, and deploy a first application and a second user or developer may develop, modify, and deploy a second application separate from a first user or developer), which may allow for focus on, and attention to, a task of a single application and/or container(s) without being hindered by tasks of another application(s) or container(s). In at least one embodiment, communication, and cooperation between different containers or applications may be aided by pipeline manager 1412 and application orchestration system 1428. In at least one embodiment, so long as an expected input and/or output of each container or application is known by a system (e.g., based on constructs of applications or containers), application orchestration system 1428 and/or pipeline manager 1412 may facilitate communication among and between, and sharing of resources among and between, each of applications or containers. In at least one embodiment, because one or more of applications or containers in deployment pipeline(s) 1410 may share same services and resources, application orchestration system 1428 may orchestrate, load balance, and determine sharing of services or resources between and among various applications or containers. In at least one embodiment, a scheduler may be used to track resource requirements of applications or containers, current usage or planned usage of these resources, and resource availability. In at least one embodiment, a scheduler may thus allocate resources to different applications and distribute resources between and among applications in view of requirements and availability of a system. In some examples, a scheduler (and/or other component of application orchestration system 1428) may determine resource availability and distribution based on constraints imposed on a system (e.g., user constraints), such as quality of service (QoS), urgency of need for data outputs (e.g., to determine whether to execute real-time processing or delayed processing), etc.

In at least one embodiment, services 1320 leveraged by and shared by applications or containers in deployment system 1306 may include compute service(s) 1416, AI service(s) 1418, visualization service(s) 1420, and/or other service types. In at least one embodiment, applications may call (e.g., execute) one or more of services 1320 to perform processing operations for an application. In at least one embodiment, compute service(s) 1416 may be leveraged by applications to perform super-computing or other high-performance computing (HPC) tasks. In at least one embodiment, compute service(s) 1416 may be leveraged to perform parallel processing (e.g., using a parallel computing platform 1430) for processing data through one or more of applications and/or one or more tasks of a single application, substantially simultaneously. In at least one embodiment, parallel computing platform 1430 (e.g., NVIDIA's CUDA) may allow general purpose computing on GPUs (GPGPU) (e.g., GPUs/Graphics 1422). In at least one embodiment, a software layer of parallel computing platform 1430 may provide access to virtual instruction sets and parallel computational elements of GPUs, for execution of compute kernels. In at least one embodiment, parallel computing platform 1430 may include memory and, in some embodiments, a memory may be shared between and among multiple containers, and/or between and among different processing tasks within a single container. In at least one embodiment, inter-process communication (IPC) calls may be generated for multiple containers and/or for multiple processes within a container to use same data from a shared segment of memory of parallel computing platform 1430 (e.g., where multiple different stages of an application or multiple applications are processing same information). In at least one embodiment, rather than making a copy of data and moving data to different locations in memory (e.g., a read/write operation), same data in same location of a memory may be used for any number of processing tasks (e.g., at a same time, at different times, etc.). In at least one embodiment, as data is used to generate new data as a result of processing, this information of a new location of data may be stored and shared between various applications. In at least one embodiment, location of data and a location of updated or modified data may be part of a definition of how a payload is understood within containers.

In at least one embodiment, AI service(s) 1418 may be leveraged to perform inferencing services for executing machine learning model(s) associated with applications (e.g., tasked with performing one or more processing tasks of an application). In at least one embodiment, AI service(s) 1418 may leverage AI system 1424 to execute machine learning model(s) (e.g., neural networks, such as CNNs) for segmentation, reconstruction, object detection, feature detection, classification, and/or other inferencing tasks. In at least one embodiment, applications of deployment pipeline(s) 1410 may use one or more of output model(s) 1316 from training system 1304 and/or other models of applications to perform inference on imaging data. In at least one embodiment, two or more examples of inferencing using application orchestration system 1428 (e.g., a scheduler) may be available. In at least one embodiment, a first category may include a high priority/low latency path that may achieve higher service level agreements, such as for performing inference on urgent requests during an emergency, or for a radiologist during diagnosis. In at least one embodiment, a second category may include a standard priority path that may be used for requests that may be non-urgent or where analysis may be performed at a later time. In at least one embodiment, application orchestration system 1428 may distribute resources (e.g., services 1320 and/or hardware 1322) based on priority paths for different inferencing tasks of AI service(s) 1418.

In at least one embodiment, shared storage may be mounted to AI service(s) 1418 within system 1400. In at least one embodiment, shared storage may operate as a cache (or other storage device type) and may be used to process inference requests from applications. In at least one embodiment, when an inference request is submitted, a request may be received by a set of API instances of deployment system 1306, and one or more instances may be selected (e.g., for best fit, for load balancing, etc.) to process a request. In at least one embodiment, to process a request, a request may be entered into a database, a machine learning model may be located from model registry 1324 if not already in a cache, a validation step may ensure appropriate machine learning model is loaded into a cache (e.g., shared storage), and/or a copy of a model may be saved to a cache. In at least one embodiment, a scheduler (e.g., of pipeline manager 1412) may be used to launch an application that is referenced in a request if an application is not already running or if there are not enough instances of an application. In at least one embodiment, if an inference server is not already launched to execute a model, an inference server may be launched. Any number of inference servers may be launched per model. In at least one embodiment, in a pull model, in which inference servers are clustered, models may be cached whenever load balancing is advantageous. In at least one embodiment, inference servers may be statically loaded in corresponding, distributed servers.

In at least one embodiment, inferencing may be performed using an inference server that runs in a container. In at least one embodiment, an instance of an inference server may be associated with a model (and optionally a plurality of versions of a model). In at least one embodiment, if an instance of an inference server does not exist when a request to perform inference on a model is received, a new instance may be loaded. In at least one embodiment, when starting an inference server, a model may be passed to an inference server such that a same container may be used to serve different models so long as inference server is running as a different instance.

In at least one embodiment, during application execution, an inference request for a given application may be received, and a container (e.g., hosting an instance of an inference server) may be loaded (if not already), and a start procedure may be called. In at least one embodiment, pre-processing logic in a container may load, decode, and/or perform any additional pre-processing on incoming data (e.g., using a CPU(s) and/or GPU(s)). In at least one embodiment, once data is prepared for inference, a container may perform inference as necessary on data. In at least one embodiment, this may include a single inference call on one image (e.g., a hand X-ray), or may require inference on hundreds of images (e.g., a chest CT). In at least one embodiment, an application may summarize results before completing, which may include, without limitation, a single confidence score, pixel level-segmentation, voxel-level segmentation, generating a visualization, or generating text to summarize findings. In at least one embodiment, different models or applications may be assigned different priorities. For example, some models may have a real-time (TAT<1 min) priority while others may have lower priority (e.g., TAT<10 min). In at least one embodiment, model execution times may be measured from requesting institution or entity and may include partner network traversal time, as well as execution on an inference service.

In at least one embodiment, transfer of requests between services 1320 and inference applications may be hidden behind a software development kit (SDK), and robust transport may be provide through a queue. In at least one embodiment, a request will be placed in a queue via an API for an individual application/tenant ID combination and an SDK will pull a request from a queue and give a request to an application. In at least one embodiment, a name of a queue may be provided in an environment from where an SDK will pick it up. In at least one embodiment, asynchronous communication through a queue may be useful as it may allow any instance of an application to pick up work as it becomes available. Results may be transferred back through a queue, to ensure no data is lost. In at least one embodiment, queues may also provide an ability to segment work, as highest priority work may go to a queue with most instances of an application connected to it, while lowest priority work may go to a queue with a single instance connected to it that processes tasks in an order received. In at least one embodiment, an application may run on a GPU-accelerated instance generated in cloud 1426, and an inference service may perform inferencing on a GPU.

In at least one embodiment, visualization service(s) 1420 may be leveraged to generate visualizations for viewing outputs of applications and/or deployment pipeline(s) 1410. In at least one embodiment, GPUs/Graphics 1422 may be leveraged by visualization service(s) 1420 to generate visualizations. In at least one embodiment, rendering effects, such as ray-tracing, may be implemented by visualization service(s) 1420 to generate higher quality visualizations. In at least one embodiment, visualizations may include, without limitation, 2D image renderings, 3D volume renderings, 3D volume reconstruction, 2D tomographic slices, virtual reality displays, augmented reality displays, etc. In at least one embodiment, virtualized environments may be used to generate a virtual interactive display or environment (e.g., a virtual environment) for interaction by users of a system (e.g., doctors, nurses, radiologists, etc.). In at least one embodiment, visualization service(s) 1420 may include an internal visualizer, cinematics, and/or other rendering or image processing capabilities or functionality (e.g., ray tracing, rasterization, internal optics, etc.).

In at least one embodiment, hardware 1322 may include GPUs/Graphics 1422, AI system 1424, cloud 1426, and/or any other hardware used for executing training system 1304 and/or deployment system 1306. In at least one embodiment, GPUs/Graphics 1422 (e.g., NVIDIA's TESLA and/or QUADRO GPUS) may include any number of GPUs that may be used for executing processing tasks of compute service(s) 1416, AI service(s) 1418, visualization service(s) 1420, other services, and/or any of features or functionality of software 1318. For example, with respect to AI service(s) 1418, GPUs/Graphics 1422 may be used to perform pre-processing on imaging data (or other data types used by machine learning models), post-processing on outputs of machine learning models, and/or to perform inferencing (e.g., to execute machine learning models). In at least one embodiment, cloud 1426, AI system 1424, and/or other components of system 1400 may use GPUs/Graphics 1422. In at least one embodiment, cloud 1426 may include a GPU-optimized platform for deep learning tasks. In at least one embodiment, AI system 1424 may use GPUs, and cloud 1426—or at least a portion tasked with deep learning or inferencing—may be executed using one or more AI systems 1424. As such, although hardware 1322 is illustrated as discrete components, this is not intended to be limiting, and any components of hardware 1322 may be combined with, or leveraged by, any other components of hardware 1322.

In at least one embodiment, AI system 1424 may include a purpose-built computing system (e.g., a super-computer or an HPC) configured for inferencing, deep learning, machine learning, and/or other artificial intelligence tasks. In at least one embodiment, AI system 1424 (e.g., NVIDIA's DGX) may include GPU-optimized software (e.g., a software stack) that may be executed using a plurality of GPUs/Graphics 1422, in addition to CPUs, RAM, storage, and/or other components, features, or functionality. In at least one embodiment, one or more AI systems 1424 may be implemented in cloud 1426 (e.g., in a data center) for performing some or all of AI-based processing tasks of system 1400.

In at least one embodiment, cloud 1426 may include a GPU-accelerated infrastructure (e.g., NVIDIA's NGC) that may provide a GPU-optimized platform for executing processing tasks of system 1400. In at least one embodiment, cloud 1426 may include an AI system(s) 1424 for performing one or more of AI-based tasks of system 1400 (e.g., as a hardware abstraction and scaling platform). In at least one embodiment, cloud 1426 may integrate with application orchestration system 1428 leveraging multiple GPUs to allow seamless scaling and load balancing between and among applications and services 1320. In at least one embodiment, cloud 1426 may tasked with executing at least some of services 1320 of system 1400, including compute service(s) 1416, AI service(s) 1418, and/or visualization service(s) 1420, as described herein. In at least one embodiment, cloud 1426 may perform small and large batch inference (e.g., executing NVIDIA's TENSOR RT), provide an accelerated parallel computing API and platform 1430 (e.g., NVIDIA's CUDA), execute application orchestration system 1428 (e.g., KUBERNETES), provide a graphics rendering API and platform (e.g., for ray-tracing, 2D graphics, 3D graphics, and/or other rendering techniques to produce higher quality cinematics), and/or may provide other functionality for system 1400.

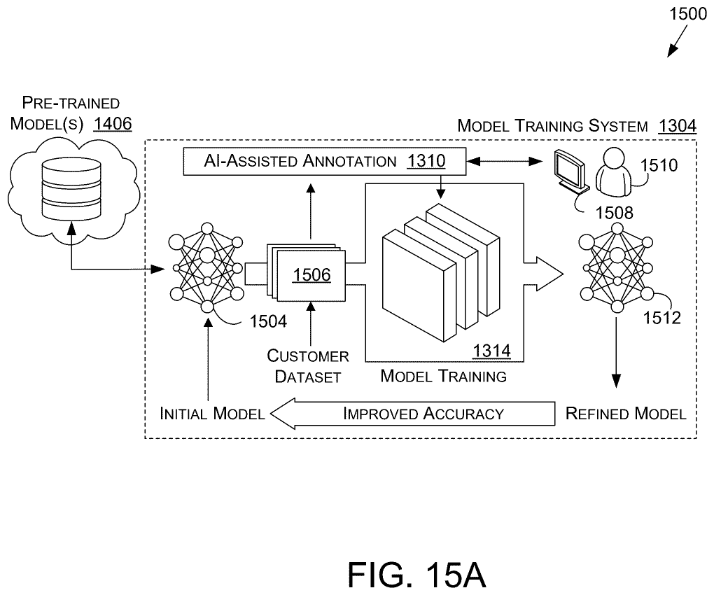

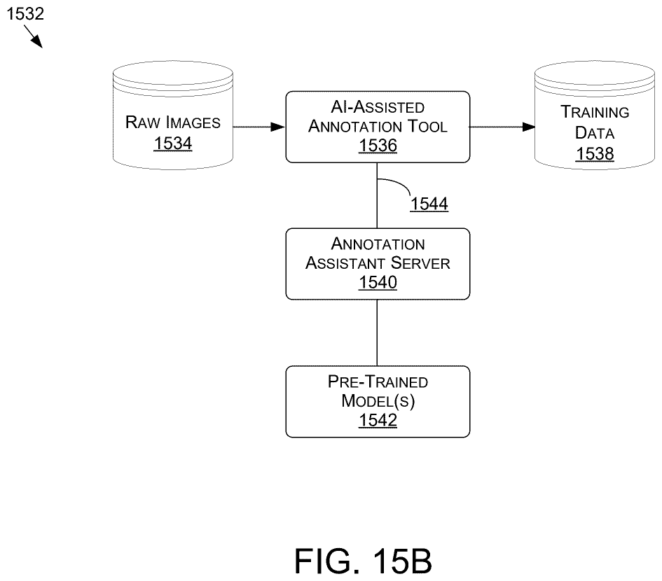

FIG. 15A illustrates a data flow diagram for a process 1500 to train, retrain, or update a machine learning model, in accordance with at least one embodiment. In at least one embodiment, process 1500 may be executed using, as a non-limiting example, system 1400 of FIG. 14. In at least one embodiment, process 1500 may leverage services and/or hardware as described herein. In at least one embodiment, refined model 1512 generated by process 1500 may be executed by a deployment system for one or more containerized applications in deployment pipelines 1510.

In at least one embodiment, model training 1514 may include retraining or updating an initial model 1504 (e.g., a pre-trained model) using new training data (e.g., new input data, such as customer dataset 1506, and/or new ground truth data associated with input data). In at least one embodiment, to retrain, or update, initial model 1504, output or loss layer(s) of initial model 1504 may be reset, deleted, and/or replaced with an updated or new output or loss layer(s). In at least one embodiment, initial model 1504 may have previously fine-tuned parameters (e.g., weights and/or biases) that remain from prior training, so training or retraining 1514 may not take as long or require as much processing as training a model from scratch. In at least one embodiment, during model training, by having reset or replaced output or loss layer(s) of initial model 1504, parameters may be updated and re-tuned for a new data set based on loss calculations associated with accuracy of output or loss layer(s) at generating predictions on new, customer dataset 1506.

In at least one embodiment, pre-trained model(s) 1506 may be stored in a data store, or registry. In at least one embodiment, pre-trained model(s) 1506 may have been trained, at least in part, at one or more facilities other than a facility executing process 1500. In at least one embodiment, to protect privacy and rights of patients, subjects, or clients of different facilities, pre-trained model(s) 1506 may have been trained, on-premise, using customer or patient data generated on-premise. In at least one embodiment, pre-trained model(s) 1506 may be trained using a cloud and/or other hardware, but confidential, privacy protected patient data may not be transferred to, used by, or accessible to any components of a cloud (or other off premise hardware). In at least one embodiment, where pre-trained model(s) 1506 is trained at using patient data from more than one facility, pre-trained model(s) 1506 may have been individually trained for each facility prior to being trained on patient or customer data from another facility. In at least one embodiment, such as where a customer or patient data has been released of privacy concerns (e.g., by waiver, for experimental use, etc.), or where a customer or patient data is included in a public data set, a customer or patient data from any number of facilities may be used to train pre-trained model(s) 1506 on-premise and/or off premise, such as in a datacenter or other cloud computing infrastructure.

In at least one embodiment, when selecting applications for use in deployment pipelines, a user may also select machine learning models to be used for specific applications. In at least one embodiment, a user may not have a model for use, so a user may select pre-trained model(s) 1506 to use with an application. In at least one embodiment, pre-trained model(s) 1506 may not be optimized for generating accurate results on customer dataset 1506 of a facility of a user (e.g., based on patient diversity, demographics, types of medical imaging devices used, etc.). In at least one embodiment, prior to deploying a pre-trained model into a deployment pipeline for use with an application(s), pre-trained model(s) 1506 may be updated, retrained, and/or fine-tuned for use at a respective facility.

In at least one embodiment, a user may select pre-trained model(s) 1506 that is to be updated, retrained, and/or fine-tuned, and this pre-trained model may be referred to as initial model 1504 for a training system within process 1500. In at least one embodiment, a customer dataset 1506 (e.g., imaging data, genomics data, sequencing data, or other data types generated by devices at a facility) may be used to perform model training (which may include, without limitation, transfer learning) on initial model 1504 to generate refined model 1512. In at least one embodiment, ground truth data corresponding to customer dataset 1506 may be generated by model training system 1304. In at least one embodiment, ground truth data may be generated, at least in part, by clinicians, scientists, doctors, practitioners, at a facility.

In at least one embodiment, AI-assisted annotation 1310 may be used in some examples to generate ground truth data. In at least one embodiment, AI-assisted annotation 1310 (e.g., implemented using an AI-assisted annotation SDK) may leverage machine learning models (e.g., neural networks) to generate suggested or predicted ground truth data for a customer dataset. In at least one embodiment, a user may use annotation tools within a user interface (a graphical user interface (GUI)) on a computing device.

In at least one embodiment, user 1510 may interact with a GUI via computing device 1508 to edit or fine-tune (auto) annotations. In at least one embodiment, a polygon editing feature may be used to move vertices of a polygon to more accurate or fine-tuned locations.

In at least one embodiment, once customer dataset 1506 has associated ground truth data, ground truth data (e.g., from AI-assisted annotation, manual labeling, etc.) may be used by during model training to generate refined model 1512. In at least one embodiment, customer dataset 1506 may be applied to initial model 1504 any number of times, and ground truth data may be used to update parameters of initial model 1504 until an acceptable level of accuracy is attained for refined model 1512. In at least one embodiment, once refined model 1512 is generated, refined model 1512 may be deployed within one or more deployment pipelines at a facility for performing one or more processing tasks with respect to medical imaging data.

In at least one embodiment, refined model 1512 may be uploaded to pre-trained models in a model registry to be selected by another facility. In at least one embodiment, this process may be completed at any number of facilities such that refined model 1512 may be further refined on new datasets any number of times to generate a more universal model.

FIG. 15B is an example illustration of a client-server architecture 1532 to enhance annotation tools with pre-trained annotation model(s) 1542, in accordance with at least one embodiment. In at least one embodiment, AI-assisted annotation tool 1536 may be instantiated based on a client-server architecture 1532. In at least one embodiment, AI-assisted annotation tool 1536 in imaging applications may aid radiologists, for example, identify organs and abnormalities. In at least one embodiment, imaging applications may include software tools that help user 1510 to identify, as a non-limiting example, a few extreme points on a particular organ of interest in raw images 1534 (e.g., in a 3D MRI or CT scan) and receive auto-annotated results for all 2D slices of a particular organ. In at least one embodiment, results may be stored in a data store as training data 1538 and used as (for example and without limitation) ground truth data for training. In at least one embodiment, when computing device 1508 sends extreme points for AI-assisted annotation, a deep learning model, for example, may receive this data as input and return inference results of a segmented organ or abnormality. In at least one embodiment, pre-instantiated annotation tools, such as AI-assisted annotation tool 1536 in FIG. 15B, may be enhanced by making API calls (e.g., API Call 1544) to a server, such as an annotation assistant server 1540 that may include a set of pre-trained model(s) 1542 stored in an annotation model registry, for example. In at least one embodiment, an annotation model registry may store pre-trained model(s) 1542 (e.g., machine learning models, such as deep learning models) that are pre-trained to perform AI-assisted annotation 1310 on a particular organ or abnormality. These models may be further updated by using training pipelines. In at least one embodiment, pre-installed annotation tools may be improved over time as new labeled data is added.

Various embodiments can be described by the following clauses:
1. A computer-implemented method, comprising:
   determining, based at least on sensor data captured using one or more sensors of a computing device, physical health data corresponding to a user of the computing device including at least one of a heart rate or a heart rate variability of the user;
   determining, using a machine learning model, a current level of anxiety of the user based at least on the physical health data; and
   adjusting, based at least on the current anxiety level of the user exceeding an anxiety threshold, one or more operational aspects of the computing device to attempt to reduce the current level of anxiety of the user.
2. The method of clause 1, further comprising:
   capturing additional sensor data of the user using one or more additional sensors of the computing device;
   determining, based at least on the additional sensor data, additional physical health data for the user; and
   determining, using the machine learning model, the current level of anxiety of the user based at least on the additional physical health data and the physical health data.
3. The method of clause 2, wherein the additional physical health data includes at least one of current blood pressure, pupil dilation, a change in voice tone or pitch, respiratory rate, a variation in user input, or a galvanic skin response.
4. The method of clause 1, wherein the one or more operational aspects include at least one of a color scheme used by a display of the computing device, a brightness of the display, a rate or type of notifications or messages indicated by the computing device, a volume or type of sound or music played, a strength or occurrence of haptic feedback, or an indication to perform one or more user-implemented mitigation actions.
5. The method of clause 1, further comprising:
   monitoring a response of the user to adjustments of the one or more operational aspects; and
   making one or more additional adjustments to the one or more operational aspects based in part on the response.
6. The method of clause 5, further comprising:
   providing data, based at least on the one or more of the response or the adjustments, as at least one of:
      additional training data for the machine learning model; or
      additional data used to create or update an anxiety profile for the user.
7. The method of clause 1, further comprising:
   allowing the user to activate anxiety monitoring and specify types or extents of adjustments that are able to be made to the one or more operational aspects in response to the current level of anxiety of the user exceeding the anxiety threshold.
8. The method of clause 1, further comprising:
   analyzing the physical health data to infer a current level of at least one of depression, stress, consciousness, or epileptic behavior.
9. The method of clause 1, wherein the method is performed using one or more interfaces exposed to one or more applications executing on the computing device and is able to adjust the one or more operational aspects of the computing device.
10. A processor, comprising:
   one or more circuits to:
      determine, based in part on sensor data of a user captured using one or more sensors of a computing device, current health data for the user;
      analyze, using a machine learning model, the current health data to determine a current level of anxiety of the user; and
      cause, based at least on the current level anxiety of the user exceeding an anxiety threshold, at least one operational aspect of the computing device to be adjusted in order to attempt to reduce the current level of anxiety of the user.
11. The processor of clause 10, wherein the current health data includes at least one of heart rate, heart rate variability, pupil dilation, response time, galvanic response, respiratory rate, voice pitch, pattern of motion, expression, or blood pressure of the user.
12. The processor of clause 11, wherein the one or more sensors include at least one of a camera, an infrared imaging sensor, a depth sensor, a motion sensor, a fingerprint scanner, a microphone, a haptic sensor, or a light sensor.
13. The processor of clause 11, wherein the at least one operational aspect includes at least one of a color scheme used by a display of the computing device, a brightness of the display, a rate or type of notifications or messages indicated by the computing device, a volume or type of sound or music played, a strength or occurrence of haptic feedback, or a signal to perform one or more user-indicated mitigation actions.
14. The processor of clause 10, wherein the one or more circuits are further to:
   monitor a response of the user to adjustments of the at least one operational aspect; and
   perform one or more additional adjustments based in part on the response.
15. The processor of clause 10, wherein the processor is comprised in at least one of:
   a system for performing simulation operations;
   a system for performing simulation operations to test or validate autonomous machine applications;
   a system for performing digital twin operations;
   a system for performing light transport simulation;
   a system for rendering graphical output;
   a system for performing deep learning operations;
   a system implemented using an edge device;
   a system for generating or presenting virtual reality (VR) content;
   a system for generating or presenting augmented reality (AR) content;
   a system for generating or presenting mixed reality (MR) content;
   a system incorporating one or more Virtual Machines (VMs);
   a system implemented at least partially in a data center;
   a system for performing hardware testing using simulation;
   a system for synthetic data generation;
   a system for performing generative AI operations using a large language model (LLM),
   a collaborative content creation platform for 3D assets; or
   a system implemented at least partially using cloud computing resources.
16. A system, comprising:
   one or more processors to determine, based at least on sensor data captured of a subject of a computing device, a current state or behavior and cause one or more mitigation mechanisms to be automatically activated on the computing device, the one or more mitigation mechanisms selected based at least on one or more of:
   the current state;
   the current behavior; or
   one or more mitigation preferences of the subject.
17. The system of clause 16, wherein the one or more mitigation preferences are specified by the subject or learned about the subject over time.
18. The system of clause 16, wherein one or more processors are located in at least one of a vehicle, a laptop computer, a gaming console, a personal computer, or a control system.
19. The system of clause 16, wherein the current state or behavior of the subject includes at least one of a state of anxiety, depression, stress, consciousness, or epileptic behavior.
20. The system of clause 16, wherein the system comprises at least one of:
   a system for performing simulation operations;
   a system for performing simulation operations to test or validate autonomous machine applications;
   a system for performing digital twin operations;
   a system for performing light transport simulation;
   a system for rendering graphical output;
   a system for performing deep learning operations;
   a system for performing generative AI operations using a large language model (LLM), a system implemented using an edge device;
   a system for generating or presenting virtual reality (VR) content;
   a system for generating or presenting augmented reality (AR) content;
   a system for generating or presenting mixed reality (MR) content;
   a system incorporating one or more Virtual Machines (VMs);
   a system implemented at least partially in a data center;
   a system for performing hardware testing using simulation;
   a system for synthetic data generation;
   a collaborative content creation platform for 3D assets; or
   a system implemented at least partially using cloud computing resources.

Other variations are within spirit of present disclosure. Thus, while disclosed techniques are susceptible to various modifications and alternative constructions, certain illustrated embodiments thereof are shown in drawings and have been described above in detail. It should be understood, however, that there is no intention to limit disclosure to specific form or forms disclosed, but on contrary, intention is to cover all modifications, alternative constructions, and equivalents falling within spirit and scope of disclosure, as defined in appended claims.

Use of terms “a” and “an” and “the” and similar referents in context of describing disclosed embodiments (especially in context of following claims) are to be construed to cover both singular and plural, unless otherwise indicated herein or clearly contradicted by context, and not as a definition of a term. Terms “comprising,” “having,” “including,” and “containing” are to be construed as open-ended terms (meaning “including, but not limited to,”) unless otherwise noted. Term “connected,” when unmodified and referring to physical connections, is to be construed as partly or wholly contained within, attached to, or joined together, even if there is something intervening. Recitation of ranges of values herein are merely intended to serve as a shorthand method of referring individually to each separate value falling within range, unless otherwise indicated herein and each separate value is incorporated into specification as if it were individually recited herein. Use of term “set” (e.g., “a set of items”) or “subset,” unless otherwise noted or contradicted by context, is to be construed as a nonempty collection comprising one or more members. Further, unless otherwise noted or contradicted by context, term “subset” of a corresponding set does not necessarily denote a proper subset of corresponding set, but subset and corresponding set may be equal.

Conjunctive language, such as phrases of form “at least one of A, B, and C,” or “at least one of A, B and C,” unless specifically stated otherwise or otherwise clearly contradicted by context, is otherwise understood with context as used in general to present that an item, term, etc., may be either A or B or C, or any nonempty subset of set of A and B and C. For instance, in illustrative example of a set having three members, conjunctive phrases “at least one of A, B, and C” and “at least one of A, B and C” refer to any of following sets: {A}, {B}, {C}, {A, B}, {A, C}, {B, C}, {A, B, C}. Thus, such conjunctive language is not generally intended to imply that certain embodiments require at least one of A, at least one of B, and at least one of C each to be present. In addition, unless otherwise noted or contradicted by context, term “plurality” indicates a state of being plural (e.g., “a plurality of items” indicates multiple items). A plurality is at least two items, but can be more when so indicated either explicitly or by context. Further, unless stated otherwise or otherwise clear from context, phrase “based on” means “based at least in part on” and not “based solely on.”

Operations of processes described herein can be performed in any suitable order unless otherwise indicated herein or otherwise clearly contradicted by context. In at least one embodiment, a process such as those processes described herein (or variations and/or combinations thereof) is performed under control of one or more computer systems configured with executable instructions and is implemented as code (e.g., executable instructions, one or more computer programs or one or more applications) executing collectively on one or more processors, by hardware or combinations thereof. In at least one embodiment, code is stored on a computer-readable storage medium, for example, in form of a computer program comprising a plurality of instructions executable by one or more processors. In at least one embodiment, a computer-readable storage medium is a non-transitory computer-readable storage medium that excludes transitory signals (e.g., a propagating transient electric or electromagnetic transmission) but includes non-transitory data storage circuitry (e.g., buffers, cache, and queues) within transceivers of transitory signals. In at least one embodiment, code (e.g., executable code or source code) is stored on a set of one or more non-transitory computer-readable storage media having stored thereon executable instructions (or other memory to store executable instructions) that, when executed (i.e., as a result of being executed) by one or more processors of a computer system, cause computer system to perform operations described herein. A set of non-transitory computer-readable storage media, in at least one embodiment, comprises multiple non-transitory computer-readable storage media and one or more of individual non-transitory storage media of multiple non-transitory computer-readable storage media lack all of code while multiple non-transitory computer-readable storage media collectively store all of code. In at least one embodiment, executable instructions are executed such that different instructions are executed by different processors—for example, a non-transitory computer-readable storage medium store instructions and a main central processing unit (“CPU”) executes some of instructions while a graphics processing unit (“GPU”) executes other instructions. In at least one embodiment, different components of a computer system have separate processors and different processors execute different subsets of instructions.

Accordingly, in at least one embodiment, computer systems are configured to implement one or more services that singly or collectively perform operations of processes described herein and such computer systems are configured with applicable hardware and/or software that enable performance of operations. Further, a computer system that implements at least one embodiment of present disclosure is a single device and, in another embodiment, is a distributed computer system comprising multiple devices that operate differently such that distributed computer system performs operations described herein and such that a single device does not perform all operations.

Use of any and all examples, or exemplary language (e.g., “such as”) provided herein, is intended merely to better illuminate embodiments of disclosure and does not pose a limitation on scope of disclosure unless otherwise claimed. No language in specification should be construed as indicating any non-claimed element as essential to practice of disclosure.

All references, including publications, patent applications, and patents, cited herein are hereby incorporated by reference to same extent as if each reference were individually and specifically indicated to be incorporated by reference and were set forth in its entirety herein.

In description and claims, terms “coupled” and “connected,” along with their derivatives, may be used. It should be understood that these terms may be not intended as synonyms for each other. Rather, in particular examples, “connected” or “coupled” may be used to indicate that two or more elements are in direct or indirect physical or electrical contact with each other. “Coupled” may also mean that two or more elements are not in direct contact with each other, but yet still co-operate or interact with each other.

Unless specifically stated otherwise, it may be appreciated that throughout specification terms such as “processing,” “computing,” “calculating,” “determining,” or like, refer to action and/or processes of a computer or computing system, or similar electronic computing device, that manipulate and/or transform data represented as physical, such as electronic, quantities within computing system's registers and/or memories into other data similarly represented as physical quantities within computing system's memories, registers or other such information storage, transmission or display devices.

In a similar manner, term “processor” may refer to any device or portion of a device that processes electronic data from registers and/or memory and transform that electronic data into other electronic data that may be stored in registers and/or memory. As non-limiting examples, “processor” may be a CPU or a GPU. A “computing platform” may comprise one or more processors. As used herein, “software” processes may include, for example, software and/or hardware entities that perform work over time, such as tasks, threads, and intelligent agents. Also, each process may refer to multiple processes, for carrying out instructions in sequence or in parallel, continuously or intermittently. Terms “system” and “method” are used herein interchangeably insofar as system may embody one or more methods and methods may be considered a system.

In present document, references may be made to obtaining, acquiring, receiving, or inputting analog or digital data into a subsystem, computer system, or computer-implemented machine. Obtaining, acquiring, receiving, or inputting analog and digital data can be accomplished in a variety of ways such as by receiving data as a parameter of a function call or a call to an application programming interface. In some implementations, process of obtaining, acquiring, receiving, or inputting analog or digital data can be accomplished by transferring data via a serial or parallel interface. In another implementation, process of obtaining, acquiring, receiving, or inputting analog or digital data can be accomplished by transferring data via a computer network from providing entity to acquiring entity. References may also be made to providing, outputting, transmitting, sending, or presenting analog or digital data. In various examples, process of providing, outputting, transmitting, sending, or presenting analog or digital data can be accomplished by transferring data as an input or output parameter of a function call, a parameter of an application programming interface or interprocess communication mechanism.

Although discussion above sets forth example implementations of described techniques, other architectures may be used to implement described functionality, and are intended to be within scope of this disclosure. Furthermore, although specific distributions of responsibilities are defined above for purposes of discussion, various functions and responsibilities might be distributed and divided in different ways, depending on circumstances.

Furthermore, although subject matter has been described in language specific to structural features and/or methodological acts, it is to be understood that subject matter claimed in appended claims is not necessarily limited to specific features or acts described. Rather, specific features and acts are disclosed as exemplary forms of implementing the claims.

What is claimed is:

1. A computer-implemented method, comprising:
determining, based at least on sensor data captured using one or more sensors of a computing device, physical health data corresponding to a user of the computing device including at least one of a heart rate or a heart rate variability of the user;
determining, using a machine learning model, a current level of anxiety of the user based at least on the physical health data; and
adjusting, based at least on the current anxiety level of the user exceeding an anxiety threshold, one or more operational aspects of the computing device to attempt to reduce the current level of anxiety of the user.

2. The method of claim 1, further comprising:
capturing additional sensor data of the user using one or more additional sensors of the computing device;
determining, based at least on the additional sensor data, additional physical health data for the user; and
determining, using the machine learning model, the current level of anxiety of the user based at least on the additional physical health data and the physical health data.

3. The method of claim 2, wherein the additional physical health data includes at least one of current blood pressure, pupil dilation, a change in voice tone or pitch, respiratory rate, a variation in user input, or a galvanic skin response.

4. The method of claim 1, wherein the one or more operational aspects include at least one of a color scheme used by a display of the computing device, a brightness of the display, a rate or type of notifications or messages indicated by the computing device, a volume or type of sound or music played, a strength or occurrence of haptic feedback, or an indication to perform one or more user-implemented mitigation actions.

5. The method of claim 1, further comprising:
monitoring a response of the user to adjustments of the one or more operational aspects; and
making one or more additional adjustments to the one or more operational aspects based in part on the response.

6. The method of claim 5, further comprising:
providing data, based at least on the one or more of the response or the adjustments, as at least one of:
additional training data for the machine learning model; or
additional data used to create or update an anxiety profile for the user.

7. The method of claim 1, further comprising:
allowing the user to activate anxiety monitoring and specify types or extents of adjustments that are able to be made to the one or more operational aspects in response to the current level of anxiety of the user exceeding the anxiety threshold.

8. The method of claim 1, further comprising:
analyzing the physical health data to infer a current level of at least one of depression, stress, consciousness, or epileptic behavior.

9. The method of claim 1, wherein the method is performed using one or more interfaces exposed to one or more applications executing on the computing device and is able to adjust the one or more operational aspects of the computing device.

10. A processor, comprising:
one or more circuits to:
determine, based in part on sensor data of a user captured using one or more sensors of a computing device, current health data for the user;
analyze, using a machine learning model, the current health data to determine a current level of anxiety of the user; and
cause, based at least on the current level anxiety of the user exceeding an anxiety threshold, at least one operational aspect of the computing device to be adjusted in order to attempt to reduce the current level of anxiety of the user.

11. The processor of claim 10, wherein the current health data includes at least one of heart rate, heart rate variability, pupil dilation, response time, galvanic response, respiratory rate, voice pitch, pattern of motion, expression, or blood pressure of the user.

12. The processor of claim 11, wherein the one or more sensors include at least one of a camera, an infrared imaging sensor, a depth sensor, a motion sensor, a fingerprint scanner, a microphone, a haptic sensor, or a light sensor.

13. The processor of claim 11, wherein the at least one operational aspect includes at least one of a color scheme used by a display of the computing device, a brightness of the display, a rate or type of notifications or messages indicated by the computing device, a volume or type of sound or music played, a strength or occurrence of haptic feedback, or a signal to perform one or more user-indicated mitigation actions.

14. The processor of claim 10, wherein the one or more circuits are further to:
monitor a response of the user to adjustments of the at least one operational aspect; and
perform one or more additional adjustments based in part on the response.

15. The processor of claim 10, wherein the processor is comprised in at least one of:
a system for performing simulation operations;
a system for performing simulation operations to test or validate autonomous machine applications;
a system for performing digital twin operations;
a system for performing light transport simulation;
a system for rendering graphical output;
a system for performing deep learning operations;
a system implemented using an edge device;
a system for generating or presenting virtual reality (VR) content;
a system for generating or presenting augmented reality (AR) content;
a system for generating or presenting mixed reality (MR) content;
a system incorporating one or more Virtual Machines (VMs);
a system implemented at least partially in a data center;
a system for performing hardware testing using simulation;
a system for synthetic data generation;
a system for performing generative AI operations using a large language model (LLM),
a collaborative content creation platform for 3D assets; or
a system implemented at least partially using cloud computing resources.

16. A system, comprising:
one or more processors to determine, based at least on sensor data captured of a subject of a computing device, a current state or behavior and cause one or more mitigation mechanisms to be automatically activated on the computing device, the one or more mitigation mechanisms selected based at least on one or more of:
the current state;
the current behavior; or
one or more mitigation preferences of the subject.

17. The system of claim 16, wherein the one or more mitigation preferences are specified by the subject or learned about the subject over time.

18. The system of claim 16, wherein one or more processors are located in at least one of a vehicle, a laptop computer, a gaming console, a personal computer, or a control system.

19. The system of claim 16, wherein the current state or behavior of the subject includes at least one of a state of anxiety, depression, stress, consciousness, or epileptic behavior.

20. The system of claim 16, wherein the system comprises at least one of:
a system for performing simulation operations;
a system for performing simulation operations to test or validate autonomous machine applications;
a system for performing digital twin operations;
a system for performing light transport simulation;
a system for rendering graphical output;
a system for performing deep learning operations;
a system for performing generative AI operations using a large language model (LLM),
a system implemented using an edge device;
a system for generating or presenting virtual reality (VR) content;
a system for generating or presenting augmented reality (AR) content;
a system for generating or presenting mixed reality (MR) content;
a system incorporating one or more Virtual Machines (VMs);
a system implemented at least partially in a data center;
a system for performing hardware testing using simulation;
a system for synthetic data generation;
a collaborative content creation platform for 3D assets; or
a system implemented at least partially using cloud computing resources.
